(19) United States
(12) Patent Application Publication
(10) Pub. No.: US 2019/0223792 A1
(43) Pub. Date: Jul. 25, 2019

(54) DRUG DELIVERY INTERGRATED NETWORK SYSTEMS

(71) Applicants: Parul Dhar, Dalla, TX (US); Bela Malik, Corpus Christi, TX (US)

(72) Inventors: Parul Dhar, Dalla, TX (US); Bela Malik, Corpus Christi, TX (US)

(21) Appl. No.: 15/879,338

(22) Filed: Jan. 24, 2018

(51) Int. Cl. A61B 5/00; A61B 5/08; A61B 5/01; A61B 5/0205; G16H 20/10

(52) U.S. Cl. CPC A61B 5/682; A61B 5/0022; A61B 5/0816; A61B 5/024; A61B 5/02055; G16H 20/10; A61B 5/01

(57) ABSTRACT

A device, system and components configured to be inserted or imbedded in an oral cavity of an animal or human for oral mucosa drug delivery system, OMDDS. The device includes a receptacle for one or more OMDDS with or without enhancer for drug delivery. The enhancer can assist in delivery via non-invasive or minimally invasive active or passive mythologies. The device is configured for several diseases including local and systemic disease. The device can replace invasive injections while reducing patient discomfort and pain. The device can be further configured to interface a plurality of one or more customizable functions and applications; including, but not limited to, auxiliary body biosensors, closed loop diagnosis and medication administration, data collections, alerting, reporting, communication network, preset biometrics and dose ranges. The system includes technology-integration, analytics, and wearables to accurately determine health referred to communication network of wearable drug delivery system.

### CROSS-REFERENCE TO RELATED APPLICATIONS

This application is continuation-in-part and claims priority to U.S. Pat. App. No. 62/180,841 entitled “Oral Biosensor Alerts and Communication System” which was filed on Jun. 17, 2015; U.S. patent application No. 14/850,713 entitled “Oral Sensor Alerting and Communication System and Developers' Tool Kit” which was filed on Sep. 10, 2015, U.S. patent application Ser. No. 15/836,901 entitled “In-situ Salivary Component Collection, Concentration, Isolation, Analytics, and Communication System” which was filed on Dec. 10, 2017, and which are incorporated herein by reference. This application claims priority to U.S. Pat. App. No. 62460978 entitled “Drug Delivery Integrated Network Systems” which was filed on Feb. 20, 2017.

### STATEMENT REGARDING FEDERALLY SPONSORED RESEARCH OR DEVELOPMENT

Not applicable.

The present disclosure relates generally to smart, oral sensors and medication administration devices and the integration of such with mobile communications, alerting and related technologies for both animals and humans, referred to herein as a DRUG DELIVERY INTERGRATED NETWORK SYSTEMS.

### BACKGROUND

background is incorporated herein by reference from the U.S. patent application Ser. No. 14/850,713 entitled “Oral Sensor Alerting and Communication System and Developers' Tool Kit,” which was filed on Sep. 10, 2015, and paragraph numbers 004-006.

Currently, drug delivery via oral tissues is limited to tables and liquids to be manually place in sublingual oral cavity for absorption which lacks efficient medication administrations due to ingestion of the medication. These pills or liquids are used without any systems to control or enhance drug delivery for both systemic and local drug administration. Furthermore, the deficiency of integration between multitudes of disciplines fails to address the growing need for the next level of metadata, drug delivery, and biological tools which could provide effective and efficient drug delivery as needed with greater accuracy as stated herein in the present invention.

### SUMMARY OF THE INVENTION

The present invention provides oral mucosal drug delivery systems for local or systemic medications which could be controlled and enhanced by one or more systems and methods described therein. Furthermore, the Oral Mucosal-Drug Delivery System (OMDDS) could be integrated with smart oral or non-oral diagnostic devices, systems and methods relating thereto, as well as auxiliary devices and methods, for greatly improving animal and human therapeutic and well-being through innovations in such technologies. The device could further configure to be smart and could contain oral mucosa drug delivery enhancing system/s. These drug delivery enhancing systems could be electrical for example, electroporation and ionophoresis, thermal such as laser, radio frequency heating, chemical heating, and mechanical heating, vibrational or ultrasound sonophoresis and phonophoresis, mechanically, e.g., chemical enhancers, and velocity assisted devices e.g. velocity based are powder injections and jet injections; mechanical assisted are micor-needles and tape-stripping, chemical enhancers such as surfactants, liposomes, microemulsion, niosomes, pronisome, carrier associate vesicles or drugs, enzyme inhibitors, nanparticles, polymer-chain associated drugs, microparticulate, mucoadhesives, diffusion, osmosis, and any combination thereof.

The invention further combines the afore mentioned device and methods with calibrations, communications, software management, data management, instant and long term animal and human analyses, multimedia inputs, visualizations, geometric motion, tracking, kinematics, alerting, therapeutic, electronic medical records, ophthalmoception, audioception, gustaoception, olfacoception or olfacception, tactioception, (thermoception), kinesthetic sense (proprioception), pain (nociception), balance (equilibrioception), vibration (mechanoreception), and various internal stimuli (e.g. the different chemoreceptors), tension sensors, pressure, stretch receptors, thirst, hunger, and time perception and other beneficial systems not previously available.

The OMDDS of the invention combines and provides for communication systems and alerting technology that links a multitude of non-oral or oral saliva-based diagnostic, therapeutic, and medication administration information inputs together. This method of integration of medication administration from oral devices and oral or non-oral diagnostics and therapeutic provide the basis for a real-time or near-time or short or long-term snapshot of effects of medication on an animal or human's health and well-being. Furthermore, these integrate disease symptoms and OMDDS in most beneficial ways for proper health of human and animal individuals.

Accordingly, an OMDDS and administration system, diagnostic oral or non-oral sensors and drug dispenser alerts and communication system, methods, and devices related to and used in conjunction therewith are provided which address the needs and provide the advantages outlined herein.

Also provided is an Oral Sensor Wearable Technology (OSWT) oral mucosal-OMDDS device according to the invention where the oral mucosa drug delivery system in combination with oral sensors and/or other sensors and devices are connected to “smart” medical devices in response to alerts and/or signals from the OSWT-oral mucosal drug delivery system.

Oral mucosal drug delivery can have several benefits when compared to traditional oral ingestion, injections, and transdermal routes. Anyone familiar with the arts understands that drug delivery through oral mucosa minimizes the role of the first-pass metabolic effects are removed, non-invasive delivery, decreased pain and increased patient compliance, decreased generation of dangerous medical sharps, and reduced risk of needle contamination, disease transmission, and needle misuse and lower cost. Oral mucosal delivery can allow for quick or sustained release profiles for both systemic and localized drug delivery. These could be painless, cause little or no bleeding, eliminate transdermal dosing variability of small molecules, the minimal possibility of pathogens introduction, can be self-administration, administered by a skilled practitioner, and reduce instances of accidental needle-stick injuries. The medication administration or drug dispensing could be connected to a secure network for a plurality of functions.

Currently, several drugs can be delivered through buccal or sublingual pathways. Such drugs are formulated in form of sprays, tablets, films, capsules, liquid, emulsions, gels, powders, etc. and deliver the drug to the local and systemic system. The effectiveness of these depend upon contact time, contact angle, rate of absorption, limited contact area, bond formation between drug molecules and oral content, etc. Often the drugs are washed away by saliva and ingested before they are absorbed, and thus reduce the drug bio-availability and effectiveness. The bio-availability can be enhanced by controlling contact time, contact angle, etc. when the drugs are inserted or placed in the oral cavity in combination with dental devices described in herein.

In one embodiment of the invention, dental devices or their attachment can contain a drug such that the contact time, angle, absorption area, absorption rate, release rate, etc. can be controlled. These, in turn, control the oral drug delivery as mentioned in herein. The amount and rate, absorption area, absorption rate, release rate, etc. of the drug delivery could be controlled via the use of a pump or an infusion pump. Furthermore, contact time, amount of contact area, contact angle, etc. can be optimized to achieve best drug delivery through the oral cavity.The angle, contact time, contact area, etc., could be controlled by device design and also could be customizable for each user. This design could be flexible or moldable to ensure efficient drug delivery. Some or all parts of the device that deliver drugs can be flexible, inflexible, fittable, moldable, adaptable, compliant, ductile, pliable, supple, tractable, tactile, transformable, workable, yielding, malleable, adjustable, changeable, conformable, convertible, bendable, and any combination thereof for most optimum drug delivery and also such that contact area and angle can be controlled either manually or automatically. Further control of the contact time, angle, area, etc., could be achieved by micro- or nano-hinges, -pivots, -hinges with micro- or nano-teeth, dial, others, or a combination thereof. The drug formulation can be formulated such that the drug release could be either fast- or slow-release as needed. The drug can face inside the dental device and contact with the gum, inner lips, checks, mouth roof, or floor. However, in some situations, the drug can face outside of the device so that it faces towards oral cavity.

In another embodiment of the invention, a custom-fitted device containing medication is worn by an individual. The medication is released when the dental device is worn by the individual; a signal can be sent to physician and others when the dental device containing the medication is worn by the individual and can prevent resale or use of the medication by people other than the prescribed patient. This embodiment has utility in prevention of resale of addictive medication in the underground market.

Ingested drugs could be monitored via communication between the dental device and sensors dispensers inside a pill or outside a pill. A pill can contain a sensor such that the sensors are released inside gut during digestion. The released sensor could communicate with the electronics on the custom-fitted dental device which in turn can send a signal to the prescribing physicians and others. Alternately, the sensor could be coating the pill such that when placed in the mouth, the sensor becomes activated and communicates with the dental device. This communication could be time and distance sensitive from the dental device such that the movement of the pill through the digestive system be tracked until it is digested and the sensor is lost.

In another embodiment of the invention, the medication is placed in the dental device such that the medication is only dispensed when the device is worn by the patient for whom the medication is prescribed. This can be accomplished by either custom-fitted device which is turned on when worn only by the patient or turned on by an authorized person or a combination of both. The drug amount and time of medication dispensed is communicated through a secure network to authorized people.

Local drug delivery can be achieved for dental or oral diseases. Currently, only teeth-whitening agents are used in conjunction with dental trays, but no such devices are used to deliver drugs. Drugs, for example, Arestin, are used in the treatment of periodontal disease are formulated to fill in the gum cavities or inserted in gum as chips or powder. Dental devices containing medications can be used to deliver drugs locally and treat dental diseases. In one embodiment of the invention, the dental device can contain medication such that the medication is in contact with diseased areas of gums, mouth roof, sublingual area, etc. When the dental device is worn, the drug is delivered to the affected area so that the disease can be treated. This device can be disposable or refillable.

Besides serving as a receptacle for drugs that can be delivered through the oral cavity, the dental devices can also contain additional devices that can assist in drug delivery. These can be electrical, thermal, vibrational or ultrasound, mechanically, chemical enhancers, and velocity assisted devices. Examples of electrically assisted are electroporation and ionophoresis, of thermally assisted are laser, radio frequency heating, chemical heating, and mechanical heating. Ultrasound assisted are sonophoresis and phonophoresis; velocity based are powder injections and jet injections; mechanical assisted are micro-needles and tape-stripping; chemical enhancers are surfactants, liposomes, microemulsion, niosomes, proniosome, carrier associate vesicles or drugs, enzyme inhibitors, nanoparticles, polymer-chain associated drugs, microparticulate, mucoadhesives, diffusion, osmosis, etc. These devices can be miniaturized to fit in the oral cavity by use of mico or nano-electronics and micro- or nano-technology.

In one embodiment of the invention, one or more devices that assist OIVIDDS in the oral cavity can be part of or attached to a dental device such that the drug can be effectively delivered in a calibrated manner. For example, an array of micro-needles containing the vaccines or peptide drugs are either attached or are part of a dental device. When an individual needs to administer a medicine that is destroyed in the gut such as vaccines, protein, or peptide drugs, insulin, and other, an array of micro-needles as a part or attached to a dental device could be inserted in the oral cavity of this individual. The array contains the drug is placed at a location within the oral cavity such that maximum drug delivery can occur. The device can be placed for sufficient amount of time to ensure proper dosage delivery. Additionally, to achieve maximum absorption the drug can be combined with chemical enhancers such as oral digestive enzyme inhibitors, mucoadhesives, or other.

Some oral mucosal drug delivery or medication administration devices selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information. These systems are used so that they cannot be swallowed.

In an aspect of the invention, a device is provided which includes an oral mucosal-drug delivery with or without sensors receptacle for the oral device. The receptacle is configured to be inserted in an oral cavity of an animal or human. The receptacle is configured to serve one or more functions within the animal or human's oral cavity wherein the one or more functions could be customized by physicians, veterinarians, patients, animal owners, users, and caretakers. The customizable functions can utilize mechatronics and can be integrated with medication administration, calibration, and sensors selected to treat, medicate, measure and diagnose one or more medical health and athletic performance biometrics and therapeutics. OSWT system imbedded oral cavity medication administration device and sensors could be utilized in conjunction with including, but not limited to, consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information. These extraction systems and sensors are used so that they cannot be swallowed. The device includes one or more medication administration systems and sensors contained within or upon the receptacle or multiple receptacles.

In another embodiment of the invention, the OSWT-OMDDS can streamline and integrate therapeutic, health, and performance measurements such as, but not limited to, various geometric models, visualization, complex spatial-temporal relations, human and animal facial and physical relationships (individually and group), data associations (i.e., pixels, auditory, motion, optimum breathing, oral air-flow, accelerometers, gyroscope, metabolic biosensors, drug dispensers, medication administration, high-definition video capture, body-wearable sensors and drug dispensers, medication administration, RFIDs, readers, positioning, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple, various degrees of dynamic alerting, tracking, positioning, multi-media, analytics, historical and other comparative data inputs, communications and platforms). Collectively, these inputs can be synced and integrated with all forms of data capture. The OSWT-oral mucosal drug delivery system can provide important real-time or near time analytics to correct or modify a motion, behavior for individuals, or organizational groups for animals and humans.

In a further embodiment, the invention provides an oral mucosal-drug delivery system including the above-described smart receptacle, one or more medications dispensers and sensors contained within, attached, or upon the receptacle and at least one interface with a network configured to utilize the information obtained from the one or more dispensers and sensors.

In an embodiment of the invention, the receptacle could be configured to contain OMDDS, OSWT-OMDDS, In-situ Saliva Component Collection, Concentration, Isolation, and Storage Communication System (ISCCCISCS), In-situ Saliva Component Collection, Concentration, Isolation, and Storage Reader System (ISCCCISRS), In-situ Blood Component Collection, Concentration, Isolation, and Storage Communication System (IBCCCISCS), Insitu Blood Component Collection, Concentration, Isolation, and Storage Reader System (IBCCCISRS), or a combination thereof In this embodiment, IBCCCISCS or IBCCCISRS could be used in combination with several trans-mucosal extractions techniques are used for trans-mucosal extractions including, but not limited to, electrically, thermal, vibrational or ultrasound, mechanically, chemical enhancers, and electrochemical gradient assisted devices. Examples of electrically assisted are electroporation, ionophoresis, and reverse iontophoresis of thermally assisted are a laser, radio frequency heating, chemical heating, and mechanical heating. Ultrasound-assisted are sonophoresis and phonophoresis; mechanical assisted are micro-needles and tape-stripping; chemical and electrochemical gradient based are salt, natural, human-made polymers and compound assisted diffusion, surfactants, enzymes, salts, chemical- or enzyme-peels, nanoparticles, polymer-chain, microparticulate, etc. It is understood by anyone familiar with arts that oral blood could substitute saliva for all embodiments and situations described herein.

It is understood by anyone familiar with the art that independent of wireless storage such that the data could be stored in any oral drug delivery device through any digital storage device, connector, or mechanism.

The invention provides, in another embodiment, a system which includes a device configured to be inserted in an oral cavity of an animal or human. The device includes a smart and calibrated drug dispenser or medication administration receptacle for one or more drug dispenser or medication administration and sensors wherein the receptacle is selected and could be customized for any human or animal condition. For example, the receptacle can be selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information. The receptacle could be configured so that it cannot be swallowed, a receptacle for babies or adults with biosensors and drug dispensers on one side and an RFID on the other side which is on the outside of a mouth, a customized teeth retainer which could be turned on when worn or when drug dispensed to enhance functionality and purpose. OSWT-oral mucosa drug delivery can include any combination of drug dispensers, biosensors, RFID tags, micro- and nano-electronics, micro- and nano-enabled energy harvesting, micro- and nano-energy storage, micro- and nano-devices, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple frequencies located in the mouth or integrated outside of a mouth. In addition, other consumer products could include a dental OSWT-oral mucosa drug delivery insert in a pet's mouth, an animal pacifier, an animal toy which could be chewed but not swallowed and fastened securely within an animal's oral cavity. The system also includes one or more sensors and drug dispensers contained within or upon the receptacle, and at least one interface with a network capable of utilizing the information obtained from the one or more sensors and drug dispensers.

Drug dispensers, sensors, and electronic can be flexible, inflexible, temporary, or permanent for analytical, other functions, and applications to provide accurate physiological information.

In yet an additional aspect, the invention includes a diagnostic or other system for an animal or human. The diagnostic or other system includes a smart, wearable or attachable device, or a device insertable to the body or other component, internal and/or external to the oral cavity. The system also includes a receptacle configured for placement within the oral cavity of the animal or human. The oral cavity receptacle is configured to serve one or more functions within the oral cavity. These include or can be selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information. These systems are used so that they cannot be swallowed.

The system also includes one or more sensors and drug dispensers located within or upon the oral cavity receptacle. The smart, wearable, attachable or externally insertable device is configured to obtain information from, provide information to, or both, the one or more sensors and drug dispensers located within or upon the oral cavity receptacle. And, the one or more sensors and drug dispensers or the smart, external device, or both, are configured to transmit the information to a network.

In yet a further embodiment of the invention, a method is provided for obtaining sensors and medication administration data from a human and/or an animal. The method includes the steps of inserting smart sensors and medication administration receptacle configured to serve one or more functions within a human or an animal's oral cavity. The smart receptacle contains or receives within or upon it one or more sensors and medication administration capable of providing information relevant to the health or a physiological characteristic of the human or animal. The method further involves activating or monitoring the one or more sensors and drug dispensers to obtain or analyze the information relevant to the health or a physiological characteristic of the human or animal and transmitting at least some portion of the health or physiological information or analysis to a network capable of utilizing the information obtained. The one or more functions of the smart sensors and drug dispensers receptacle is configured to serve, e.g., is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function for infants, children, elderly, and animals, providing sleep-aid and preventing snoring and obstructive sleep apnea function, therapeutic, providing a recreational, athletic performance, or sports function, physiological, natural and/or grinding protective function, horse-rider navigation and communication function, vital sign measuring function, diagnostic and therapeutic function, and surgical equipment and hospitalization equipment function.

In an embodiment of the invention, a method is provided for placing in the receptacle the OMDDS, OSWT-OMDDS, In-situ Saliva Component Collection, Concentration, Isolation, and Storage Communication System (ISCCCISCS), In-situ Saliva Component Collection, Concentration, Isolation, and Storage Reader System (ISCCCISRS), In-situ Blood Component Collection, Concentration, Isolation, and Storage Communication System (IBCCCISCS), Insitu Blood Component Collection, Concentration, Isolation, and Storage Reader System (IBCCCISRS), or a combination thereof.

In another embodiment, a method of using trans-mucosal extractions techniques for IBCCCISCS or IBCCCISRS including, but not limited to, electrical, thermal, vibrational or ultrasound, mechanically, chemical enhancers, and electrochemical gradient assisted devices. Examples of electrically assisted are electroporation, ionophoresis, and reverse iontophoresis of thermally assisted are a laser, radio frequency heating, chemical heating, and mechanical heating. Ultrasound-assisted are sonophoresis and phonophoresis; mechanical assisted are micro-needles and tape-stripping; chemical and electrochemical gradient based are salt, natural, human-made polymers and compound assisted diffusion, surfactants, enzymes, salts, chemical- or enzyme-peels, nanoparticles, polymer-chain, microparticulate, etc.

In an embodiment of the invention, a method is provided for networking the OMDDS, OSWT-OMDDS, In-situ Saliva Component Collection, Concentration, Isolation, and Storage Communication System (ISCCCISCS), In-situ Saliva Component Collection, Concentration, Isolation, and Storage Reader System (ISCCCISRS), In-situ Blood Component Collection, Concentration, Isolation, and Storage Communication System (IBCCCISCS), Insitu Blood Component Collection, Concentration, Isolation, and Storage Reader System (IBCCCISRS), or a combination thereof. The method further involves activating or monitoring the one or more sensors within ISCCCISC, ISCCCISRS, IBCCCISC, IBCCCISRS, and drug dispensers to obtain or analyze the information relevant to the health or a physiological characteristic of the human or animal and transmitting at least some portion of the health or physiological information or analysis to a network capable of utilizing the information obtained.

Also provided is a customizable development tool kit or platform for multiple OSWT-OMDDS purposes and functions and for building a diagnostic or other system to provide information such as, but not limited to hardware, and software integration, analysis, or alerts, for an animal, animals, human or humans. The kit includes customizable components to meet the needs of a consumer or user of the diagnostic or other system. The components include at least one sensors and calibrate-able drug dispensers insertable or temporarily or permanently imbedded within or upon one or more oral cavity receptacles, and at least one oral cavity receptacle configured for placement within the oral cavity of the animal, animals, human or humans, at least one smart auxiliary device which is wearable, attachable or insertable externally to the oral cavity. The tool kit also includes at least one network unit configured to receive information from the at least one smart, auxiliary device, the at least one sensors and drug dispensers, or both, and analyze, transmit or both, the information received. The components for selecting the at least one auxiliary device, the at least one oral cavity sensors and drug dispensers receptacle, the at least one oral cavity sensors and drug dispensers, and the at least one network unit are made available to the consumer or user to construct or have constructed a diagnostic or oral drug delivery or other system configured to obtain information, analysis or alerts customized to meet the specific needs of the consumer or user.

### BRIEF DESCRIPTION OF THE DRAWINGS

The presently disclosed subject matter will be better understood from reading the following description of non-limiting embodiments, with reference to the attached drawings, wherein below:

FIG. 1 is a schematic depiction which exemplifies an OSWT-Oral Mucosa Drug Delivery System secure wireless communication capability and information retrieval through the animal's or human's oral cavity according to the invention.

FIG. 2 is a schematic depiction showing which oral cavity tissue or portions could be most useful for oral mucosa drug delivery systems. Here we show a diagram with different parts of oral cavity. Some parts are better for OMDDS than others parts of the oral cavity.

FIG. 3 is a representative of cellular structure of oral mucosa: The oral mucosa is of two types, keratinized and non-keratinized. Areas that are subjected to mechanical stress are keratinized and are similar to the epidermis of the skin. Areas that are not subjected mechanical stress are not keratinized and unlike skin form less barrier for diagnostic measurements.

FIG. 4 depicts a schematics depiction that exemplifies the structure of an oral mucosa drug delivery device along with the enhancers and biosensors, forming OSWT-OMDDS, as may be used in embodiments of the invention.

FIG. 5 depicts a schematics depiction that exemplifies OMDDS in contact with oral mucosa drug along, as may be used in embodiments of the invention.

FIG. 6A depicts embodiments which exemplify the locations of OMDDS on a retainer.

FIG. 6B depicts embodiments which exemplify the locations of OMDDS on a mouth guard.

FIG. 7 depicts embodiments for various passive and active technologies to enhance drug delivery through the oral cavity.

FIG. 8 depicts embodiments which exemplify OSWT-OMDDS dynamic alerting software and secure networks, in accordance with embodiments of the present invention.

### DETAILED DESCRIPTION OF THE INVENTION

The foregoing summary, as well as the following detailed description of certain embodiments will be better understood when read in conjunction with the appended drawings. As used herein, an element or step recited in the singular and proceeded with the word “a” or “an” should be understood as not excluding the plural of said elements or steps, unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

As used herein, the term “smart” means a device or object that performs one or more functions of a computer or information system, such as data storage, calculation, Internet access and information transmission.

As used herein the terms “insertable”, fitable, “implantable”, “imbeddable”, “embeddable”, “temporarily insertable” “permanently insertable”, “temporarily implantable”, “permanently implantable” , “temporarily imbeddable”, “permanently imbeddable”, “temporarily embeddable” and “permanently embeddable” refer to means of securely inserting and attaching in or to, or fastening a device, such as being adhered to, cemented, affixed or otherwise securely attached to a surface or object.

As used herein, the term “receptacle” refers to a device or container that receives, retains, has within, or holds something.

Described in its broader respects, the Oral Mucosal-Drug Delivery System (OMDDS) of the invention includes a device configured to be inserted and securely attached or fitted in an oral cavity of an animal or human. The device includes medication administration or drug dispenser's receptacle for drug dispensing.

The techniques that are used for transdermal drug delivery such are iontophoresis, electroporation, reverse iontophoresis, ultrasound, sonophororesis, phonophoresis, velocity based such as powder and jet injections, laser, radio frequency heating, micro-needle, tape stripping, etc. are better suited to work in the oral cavity mucosa. The device could further configure to be smart and could contain oral drug delivery enhancing system. These drug delivery enhancing systems could be electrical, electroporation and ionophoresis; thermal, laser, radio frequency heating, chemical heating, and mechanical heating; vibrational or ultrasound sonophoresis and phonophoresis; mechanically, chemical enhancers, and velocity assisted devices, velocity based are powder injections and jet injections; mechanical assisted are micro-needles and tape-stripping; chemical enhancers are surfactants, liposomes, microemulsion, niosomes, proniosome, carrier associate vesicles or drugs, enzyme inhibitors, nanoparticles, polymer-chain associated drugs, microparticulate, mucoadhesives, diffusion, osmosis, etc.

Drug administration routes are typically of two major types, non-invasive and invasive. Non-invasive can be oral, topical, and inhalation or nasal and invasive can be injections through intravenous (IV), intramuscular (IM), subcutaneous (SC), and epidural; Disadvantages of injections include potential pain or discomfort for the patient, and the requirement of trained staff using aseptic techniques for administration. The oral route, on the other hand, is the most convenient without the need for trained staff and carries the lowest cost. Oral administration is through enteral/enteric, gastric or gastrointestinal, sublingual (under the tongue) and sublabial or buccal. Some drugs can cause gastrointestinal tract irritation or can be destroyed by the gastric environment, called first-pass metabolic effects. Sublingual and sublabial routes do not encompass first-pass metabolic effects and can mimic invasive injection route without the invasive nature of the administration route. Here we also propose administration through insertion or embedding in the oral cavity which is same as intravenous injections.

The receptacle could be preferably configured to serve one or more functions within the animal or human's oral cavity without being swallowed. The device also includes one or more drug dispensers contained within or upon the receptacle, and also at least one interface with a network configured to utilize the information obtained from the one or more drug dispensers or from one or more platforms providing additional information or capabilities networked with the system.

The system as described above may provide, e.g., one or more functions of the device including or selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function for infants, children, elderly, and animals, providing sleep-aid and preventing snoring and obstructive sleep apnea function, therapeutic, providing a recreational, athletic performance, or sports function, physiological, natural and/or grinding protective function, horse-rider navigation and communication function, vital sign measuring function, diagnostic and therapeutic function, and surgical equipment and hospitalization equipment function, health analytics, diagnostic analytics, performance analytics; integration of body sensors and drug dispensers, health-devices, nano-particles, and sports and performance sensors and drug dispensers and drug dispensers on inanimate objects and sports equipment; customizable developers' tool kit for biosensors and drug dispensers, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors and drug dispensers in order to produce computer-generated precise movement and greater precision or analytics.

In certain embodiments, the system is set up wherein at least one of the one or more functions of the device is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information. These systems are used so that they cannot be swallowed.

Additionally, the system may further include one or more drug dispensers contained within or upon one or more receptacles located within or upon the animal or human's body networked with the oral cavity or non-oral cavity device/s.

Oral mucosal drug delivery can have several benefits when compared to traditional oral ingestion, injections, and transdermal routes. Orally ingested drugs when reach stomach where several digestive enzymes can destroy the medicine molecules. Anyone familiar with the arts understands that drug delivery through oral mucosa minimizes the role of stomach digestive enzymes and low pH that can cause degradation or denature of macromolecules, so the first-pass metabolic effects are removed. Furthermore, advantages of oral mucosal delivery over hypodermic injections include, non-invasive delivery, decreased pain and increased patient compliance, decreased generation of dangerous medical sharps, and reduced risk of needle contamination, disease transmission, and needle misuse. Oral mucosal delivery can allow for quick or sustained release profiles for both systemic and localized drug delivery, which is not always possible with ingestable or injectable formulations.

Currently, several drugs can be delivered through buccal or sublingual pathways. Such drugs are formulated in form of sprays, tablets, films, capsules, liquid, emulsions, gels, powders, etc. and deliver drug to local and systemic system. The effectiveness of these depend upon contact time, contact angle, rate of absorption, limited contact area, bond formation between drug molecules and oral content, etc. Often the drugs are washed away by saliva before they are absorbed, and thus reduce the drug bio-availability and effectiveness. The bio-availability can be enhanced by controlling contact time, contact angle, etc. when the drug is part of a dental device as explained herein.

In one embodiment of invention, dental devices or their attachment can contain a drug so that the contact time, angle, area, absorption, etc. can be controlled. The contact can be made between the drug and buccal area for controlled absorption due to better muco-adhesion but the contact can be between drug and sublingual area for quick absorption due to higher sublingual surface permeability. The drug formulation can be either fast or slow release as needed.

In another embodiment of the invention, OMDDS is controlled by modulating contact time, amount of contact area, contact angle, etc. can be optimized to achieve best drug delivery through oral cavity. The device, for example, can increase contact area and increase drug release time such that larger amount is drug is delivered and lesser amount, if any, is washed by saliva and ingested making these oral drug delivery system more efficient.

In one embodiment of the invention, dental devices or their attachment can contain a drug such that the contact time, angle, absorption area, absorption rate, release rate, etc. can be controlled. These, in turn, control the oral drug delivery as mentioned in herein. The amount and rate, absorption area, absorption rate, release rate, etc. of the drug delivery could be controlled via the use of a pump or an infusion pump. Furthermore, contact time, amount of contact area, contact angle, etc. can be optimized to achieve best drug delivery through the oral cavity.The angle, contact time, contact area, etc., could be controlled by device design and also could be customizable for each user. This design could be such that some or all parts of the device that deliver drugs can be flexible, inflexible, fittable, moldable, adaptable, compliant, ductile, pliable, supple, tractable, tactile, transformable, workable, yielding, malleable, adjustable, changeable, conformable, convertible, bendable, and any combination thereof for most optimum drug delivery and also such that contact area and angle can be controlled either manually or automaticallyto ensure efficient drug delivery. Further control of the contact time, angle, area, etc., could be achieved by micro- or nano-hinges, -pivots, -hinges with micro- or nano-teeth, dial, others, or a combination thereof. The drug formulation can be formulated such that the drug release could be either fast- or slow-release as needed. The drug can face inside the dental device and contact with the gum, inner lips, checks, mouth roof, or floor. However, in some situations, the drug can face outside of the device so that it faces towards oral cavity.

In another embodiment of the invention, a custom-fitted device containing medication is worn by an individual. The medication is released when the dental device is worn by the individual; a signal or alert can be sent to physician and other when the dental device containing the medication is worn by the individual. This embodiment can prevent resale or use of the medication by people other than the prescribed patient. This has utility in prevention of resale of addictive medication in the underground market.

Local drug delivery can be achieved for dental or oral diseases. Currently, only teeth-whitening agents are used in conjunction with dental trays, but no such devices are used to deliver drugs. Drugs, for example, Arestin, are used in the treatment of periodontal disease are formulated to fill in the gum cavities or inserted in gum as chips or powder. Dental devices containing drugs can be used to deliver drugs locally and treat dental diseases. In one embodiment of the invention, the dental device can contain medication such that they make contact with diseased gums, mouth roof, sublingual area, etc. When the dental device is worn, the drug is delivered to the affected area so that the disease can be treated. This device can be disposable or refillable.

Besides serving as a receptacle for drugs that can be delivered through oral cavity, the dental devices can also contain additional devices that can enhance and assist in drug delivery. These can be electrical, thermal, vibrational or ultrasound, mechanically, chemical enhancers, and velocity assisted devices. Examples of electrically assisted are electroporation and ionophoresis, of thermally assisted are a laser, radio frequency heating, chemical heating, and mechanical heating. Ultrasound-assisted is sonophoresis and phonophoresis; velocity based are powder injections and jet injections; mechanical assisted are micro-needles and tape-stripping; chemical enhancers are surfactants, liposomes, microemulsion, niosomes, proniosome, carrier associate vesicles or drugs, enzyme inhibitors, nanoparticles, polymer-chain associated drugs, microparticulate, mucoadhesives, diffusion, osmosis, etc. These devices can be miniaturized to fit in the oral cavity by use of micro-electronics and nanotechnology.

In one embodiment of the invention, one or more devices that assist drug delivery in the oral cavity can be part of or attached to a dental device such that the drug can be effectively delivered. For example, an array of micro-needles containing the vaccines or peptide drugs are either attached or are part of a dental device. When an individual need to be administered a drug that is destroyed in the gut such as vaccines, protein, or peptide drugs, insulin, and other, an array of micro-needles as a part or attached to a dental device could be inserted in the oral cavity of the individual. Micro-needles are multiple microscopic projections typically assembled on one side of a supporting base or patch, generally ranging from 25 to 2000 μm in height, 50 to 250 μm in base width and 1 to 25 μm in tip diameter. They are painless, causes no or little bleeding, eliminate dosing variability of small molecules, the minimal possibility of pathogens introduction, can be self-administration, and reduce instances of accidental needle-stick injuries. These arrays can be an alternative or replacement for hypodermic and subcutaneous needle technologies, and they combine the ease of use of a trans-mucosal patch with the effectiveness of conventional hypodermic needle and syringes. The needles should be of a suitable length, width, shape, and placement to avoid nerve contact when inserted into skin layers to avoid pain. They are usually designed in arrays in order to improve the surface contact with the oral tissue and facilitate penetration of therapeutic molecules into the oral blood system. They are designed to create transient aqueous conduits of micron dimensions across the oral mucosa. Since these micropores are orders of magnitude larger than macro-molecular drug dimensions, they readily permit the transport of hydrophilic macromolecules. Therefore, they enhance the flux of the molecules ranging from small hydrophilic molecules such as alendronate to macromolecules such as small weight molecules heparins, insulin, and vaccines. The array contains the drug is placed at a location within the oral cavity such that maximum drug delivery can occur. The device can be placed for sufficient amount of time to ensure proper dosage delivery. Additionally, to achieve maximum absorption the drug can be combined with chemical enhancers such as oral digestive enzyme inhibitors, mucoadhesives, or others.

In one embodiment, iontophoresis is used to enhance oral drug delivery. Iontophoresis involves the application of physiologically acceptable electrical currents (0.1-1.0 mA/cm2) to drive charged molecules into the oral tissue by electrostatic effects, and hence, ionic drugs can pass through the oral mucosa into the body by the created potential gradient. Unlike other trans-mucosal enhancement methodologies, it acts mainly by involving a second driving force, the electrical potential gradient as a companion to the concentration gradient across the oral mucosa since uncharged species can also be delivered by electro-osmosis.

In another embodiment of the invention, high-velocity jets can be used to enhance oral drug delivery. These employ a high-velocity jet with velocities ranging from 100 to 200 m/s to puncture the oral mucosa and deliver drugs using a power source which is either a compressed gas or spring. There are two types of liquid jet injector single-dose jet injectors (disposable cartridge jet injectors) and multi-use-nozzle jet injectors. A jet injector is a needle-free device capable of delivering electronically controlled doses of medication, and they result in improved delivery consistency and reduced pain for the patient. Liquid-jet injectors propel liquid from a nozzle with an orifice diameter, smaller than the standard needle of 810 ranging from 50 to 360 μm. By changing the jet velocity and orifice diameter, the jet can deliver the drug into several layers of oral tissue. Powder jet injectors can deliver solid drugs or vaccines to the oral tissue which increases the stability of the formulation, and thus, removes the need for cold storage. Powder jet injectors may be formulated from nano-or micro-particles containing the active or lyophilized drugs and antigens. Excellent bioavailability for some drugs has been reported, but the intermittent pain and bruising caused to patients have restricted wide acceptance of jet injectors.

In one embodiment of the invention, low-frequency ultrasound can be used to enhance drug delivery into oral tissue. Low-frequency ultrasound can potentiate delivery of wide range of therapeutic compounds such as protein-based drugs, vaccines, gene therapy, hormones, liposomes, nano-particles, immunization adjuvant, hydrophilic molecules, and macromolecules such as interferon y (17,000 Da) and erythropoietin (48,000 Da). Low-frequency sonophoresis offers advantages over other oral mucosa delivery methods because it can be controlled by varying the application time and other ultrasound parameters. The size of aqueous mucosal pores thus created can be controlled by varying the frequency and the intensity of the ultrasound. Low-frequency ultrasound can be used alone or in combination with other enhancers by the pretreatment of other formulation followed ultrasound application.

Monitor ingested drugs via communication between dental device and sensors inside a pill or outside a pill. A pill can contain sensors such that the sensor is released into gut during digestion. This released sensor can communicate with the electronics on the custom-fitted dental device which in turn can send a signal to the prescribing physicians and others. Alternately, the sensor could be coating the pill such that when placed in the mouth, the sensor becomes activated and communicates with the dental device. This communication could be time and distance from the dental device sensitive such that the movement of the pill through the digestive system be tracked until it is digested and sensor lost.

In another embodiment of the invention, the medication is placed in the dental device such that the medication is only dispensed when the device is worn by the patient for whom the medication is prescribed. The drug amount and time of medication dispensed is communicated through a secure network to authorized people.

As to particular examples of receptacles useable for the system, these include, but are not limited to one or more of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information, and an RFID, micro- and nano-sensors and drug dispensers, micro- and nano-electronics, micro- and nano-enabled energy harvesting, micro- and nano-energy storage, micro- and nano-devices micro- and nano-programmable processors, micro- and nano-memory devices, micro- and nano-integrated power management devices, micro- and nano-programmable hardware, micro- and nano-wireless devices with communication capabilities across multiple frequencies located in an oral cavity or integrated outside of the oral cavity, and a device configured to be inserted into an animal or human's oral cavity, but not swallowed, one or more sensors and drug dispensers contained within or upon the receptacle, and at least one interface with a network capable of utilizing the information obtained from the one or more sensors and drug dispensers, one or more platforms, or one or more auxiliary devices or body integrations.

Specific examples of the system include: the smart sensors and drug dispenser receptacle is a retainer and the smart sensors, and drug dispensers receptacle is further configured with WiFi connectivity, the system includes one or more sensors and drug dispensers, and the system further provides an alerting signal when outside a pre-set range; the smart sensors and drug dispensers receptacle is a retainer with full connectivity, the system further includes full server access and is configured for an analytical processing capability; the smart sensors and drug dispensers receptacle is a full or partial retainer, devices used during surgery and hospitalization for example anesthetic machine for continuous-flow, vaporizer, oxygen mask, nasal oxygen mask, Guedel airways, oral Suction catheter, ventilation, respiratory maintenance circuit, Laryngoscope, Tracheal tube, Laryngeal mask airway, tracheostomy tube, endoscopes, eschmann stylet or gum elastic bougie, air-filters, mucus sucker, the system further includes a smart mouth guard accessory, the one or more sensors and drug dispensers includes sensors for temperature or oxygen levels, the system is further configured with WiFi connectivity and is configured to provide an alerting signal when outside a pre-set range; the smart sensors and drug dispensers receptacle is a full or partial retainer, the system further includes a smart mouth guard accessory, the system is provided with full connectivity, full server access and is configured for an analytical processing capability comprising racing performance analysis.

The network units of the system include ones capable of utilizing the information obtained from the one or more sensors and drug dispensers and having functions including, but not limited to, data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, analyzing historical or previous data from same or different individual or team, visualizing or presenting development platforms.

While there is a focus on the sensors and drug dispensers insertable, fitable, and securely attachable in the oral cavity, the system can, and in many embodiments, does include at least one auxiliary sensors and drug dispenser receptacle selected from, but not necessarily limited to, the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information. These OMDDS and sensors are used so that they cannot be swallowed, a receptacle for babies or adults with biosensors, drug dispensers, sensors, cameras, audio speakers, an RFID inside or outside of a mouth, micro- and nano-electronics, micro- and nano-enabled energy harvesting, micro- and nano-energy storage, micro- and nano-device, micro- and nano-timer, micro- and nano-programmable processors, micro- and nano-memory, micro- and nano-integrated power management, micro- and nano-programmable hardware, micro- and nano-wireless communication capabilities across multiple frequencies, sensors and drug dispensers located in a mouth or integrated outside of a mouth, and an animal toy which is configured to be sucked but not swallowed.

The system includes one or more network units which can be configured to carry out a functionality including or consisting of signaling bi-directional transmissions to a secure server through one or more of WiFi, Bluetooth, GPS, and NFC, temporarily storing information in the smart device, and bi-directionally transmitting alerts to pre-selected devices or pre-selected personnel. Further network units employable in the system include or consist of, one or more RFID components, one or more cloud applications, a real-time or near-time slumber to alert mode, a manual control diagnosis mode, a programmed automated diagnosis mode, a geographic analysis mode, a species classification analysis mode, a disease specific or situational alerting mode, a function by which the one or more oral sensors and oral drug dispensers is activated and inactivated by another sensors and drug dispenser, device or remote controller, and transmission through WiFi or other wireless mode.

Also provided is a system that comprises of a device configured to be inserted in an oral cavity of an animal or human including a smart sensors and drug dispenser receptacle for one or more sensors and drug dispensers. The receptacle can be, but is not necessarily limited to, of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information, other bio-sensors and drug dispensers, a receptacle configured to be inserted but not swallowed, a receptacle for infants or adults with biosensors and drug dispensers on one side and an RFID on the other side which is on the outside of a mouth, micro- and nano-sensors and drug dispensers located in a mouth or outside of a mouth, and a receptacle configured to be insert but not swallowed within an animal's oral cavity. The system includes drug dispensers contained within or upon the receptacle. And the system includes at least one interface with a network capable of utilizing the information obtained from the one or more drug dispensers, and sensors or from one or more platforms or one or more body integrations. The network capable of utilizing the information obtained from the one or more drug dispensers or sensors includes one or more units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, comparative, historical or previous from same or different individual or team, data producing, visualizing or presentation development platforms.

In an embodiment of the invention, the receptacle could be configured to contain OMDDS, OSWT-OMDDS, In-situ Saliva Component Collection, Concentration, Isolation, and Storage Communication System (ISCCCISCS), In-situ Saliva Component Collection, Concentration, Isolation, and Storage Reader System (ISCCCISRS), In-situ Blood Component Collection, Concentration, Isolation, and Storage Communication System (IBCCCISCS), Insitu Blood Component Collection, Concentration, Isolation, and Storage Reader System (IBCCCISRS), or a combination thereof. In this embodiment, IBCCCISCS or IBCCCISRS could be used in combination with several trans-mucosal extractions techniques are used for trans-mucosal extractions including, but not limited to, electrically, thermal, vibrational or ultrasound, mechanically, chemical enhancers, and electrochemical gradient assisted devices. Examples of electrically assisted are electroporation, ionophoresis, and reverse iontophoresis of thermally assisted are a laser, radio frequency heating, chemical heating, and mechanical heating. Ultrasound-assisted are sonophoresis and phonophoresis; mechanical assisted are micro-needles and tape-stripping; chemical and electrochemical gradient based are salt, natural, human-made polymers and compound assisted diffusion, surfactants, enzymes, salts, chemical- or enzyme-peels, nanoparticles, polymer-chain, microparticulate, etc. It is understood by anyone familiar with arts that oral blood could substitute saliva for all embodiments and situations described herein.

The system including the auxiliary drug dispenser receptacle and smart sensors can also include a network including one or more RFID components, micro- and nano-electronics, micro- and nano-enabled energy harvesting, micro- and nano-energy storage, micro- and nano-devices, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple frequencies located in a mouth or integrated outside of a mouth, one or more cloud applications, a real-time or near-time slumber to alert mode, a manual control diagnosis mode a programmed automated diagnosis mode, a geographic analysis mode, a species classification analysis mode, a disease specific or situational alerting mode.

The system can include one or more medical devices or medication dispensers. Further enhancements include a fully integrated treatment facility, a system in which one or more drug dispensers and sensors is activated by another sensors and drug dispenser, device or remote controller, network modes including transmission through WiFi or other wireless modes, and systems in which at least one auxiliary smart sensors and drug dispenser receptacle for a drug dispenser and sensors is configured to serve one or more secondary functions within the animal or human's oral cavity.

In a further aspect of the invention, applicants have provided a device configured to be inserted and securely attached or fitted in an oral cavity of an animal or human. The device includes a smart sensor and drug dispenser receptacle for sensors and drug dispenser, and the receptacle is configured to serve one or more functions within the animal or human's oral cavity without being swallowed. The device also includes one or more sensors and drug dispensers contained within or upon the receptacle.

The one or more functions served by the receptacle of the device can include or consist of functions selected from the group consisting of, but not limited to, of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function for infants, children, elderly, and animals, providing sleep-aid and preventing snoring and obstructive sleep apnea function, therapeutic, providing a recreational, athletic performance, or sports function, physiological, natural and/or grinding protective function, horse-rider navigation and communication function, vital sign measuring function, diagnostic and therapeutic function, and surgical equipment and hospitalization equipment function, providing a recreational or sports function, health analytics, diagnostic analytics, performance analytics; integration of body sensors and drug dispensers, health-devices, nano-particles, and sports and performance sensors and drug dispensers on inanimate objects and sports equipment; customizable developers' tool kit for biosensors, sensors and drug dispensers, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors and drug dispensers in order to produce computer-generated precise movement and greater precision and analytics.

In certain embodiments, the above device serves at least to provide one of the following functions: replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function for infants, children, elderly, and animals, providing sleep-aid and preventing snoring and obstructive sleep apnea function, therapeutic, providing a recreational, athletic performance, or sports function, physiological, natural and/or grinding protective function, horse-rider navigation and communication function, vital sign measuring function, diagnostic and therapeutic function, and surgical equipment and hospitalization equipment function. For example anesthetic machine for continuous-flow, vaporizer, oxygen mask, nasal oxygen mask, Guedel airways, oral Suction catheter, ventilation, respiratory maintenance circuit, Laryngoscope, Tracheal tube, Laryngeal mask airway, tracheostomy tube, endoscopes, eschmann stylet or gum elastic bougie, air-filters, mucus sucker.

Examples of particular devices embodying the invention include wherein at least one of the functions includes replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information.

In describing the nature of certain of the device receptacles which often have a conventional dental device aspect to their functionality, the receptacle can include replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information, and any combination thereof.

The device receptacle is insertable, e.g., by micro- and nano-clips, frames, brackets, sealants, dental composites, bonds, adhesives, adhesive strips, cements, wires, bands, glues, embedment, injection, printing, tattooing, micro- or nano-hinges, micro- or nano-dial with or without micro- or nano teeth, or any combination thereof.

The device can be configured to be manually, automatically, or wireless turned off or on. The position of the device can be adjusted for controlled and efficient drug delivery.

In these devices, the receptacle can be configured to be removable, self-removable, self-installable, coverable with an air-tight material, and configured so that the one or more sensors and drug dispensers is integral to the receptacle.

The sensors and drug dispensers in the provided devices can include, e.g., drugs in any physical form such as liquid, solid, aerosol, gas, chemical, protein, enzyme, biologics, nucleotide, DNA or RNA, proteins, immunoglobins, antibodies, histones, peptides, modified proteins, neuro-peptides, pigments, dissolved gases, alcohol or alcohol-containing mixtures, lipids profile, salts, metabolites, anions, cations, water, sugar, lectin, and can be a combination of two or more forms.

The drug dispensers in the provided devices can include drug for treatment of one or more systemic or local diseases including, but not limited to, systemic or localized cancer of any phase, an oral or systemic infectious disease, drug abuse, metabolic disease, malnutrition, obesity, cardiovascular disease, atherosclerotic, infection, auto-immune and other immune diseases, stroke, AIDs, multiple sclerosis, periodontal diseases, brain-function disorders, dementia, memory loss, depression, mental disease, Alzheimer's disease, mentally-challenged disorders, nervous system disorders, and other psychology and neurological disorders, bleeding, head, neck, and other injuries, Sjogen's syndrome, oxidative stress, allergies, cancer, skeletal and muscle diseases, genetic diseases, renal diseases, osteoporosis, fatigue, stress, sleep deprivation or sleep apnea, fertility, pregnancy, ovulation, and reproductive system disorders, cystic fibrosis, respiratory or pulmonary diseases, diabetes and ketoacidosis, inflammation, age-related diseases, dehydration, halitosis, alcoholism or drug abuse, consumption or drug addiction, hypoxia, smoking-related diseases, toxins, or pollutants, poor-gait, Crohn's disease, Cushing disease, dental caries, blood and circulatory disorders, ear, nose, and throat diseases, taste, Ageusia, Hypogeusia, or Dysgeusia, bad-breath related diseases, chewing or mastication related diseases, digestive disorders, hepatic diseases, spleen, gall-bladder and pancreatic diseases, urinary system disorders, integumentary system diseases, endocrine, lymphatic, and excretory diseases, anti-pathogen or anti-microbe, and any combination thereof.

The device as herein described can be securely attached or fitted within the oral cavity of an animal or human by a number of means, including one or more of being fixedly inserted, imbedded, fitted, fixed, implanted, fastened, joined, associated, coupled, linked, banded, united, mounted, combined, glued, adhered, cemented, or firmly connected by mouth, e.g., lips, teeth, etc., or hands or parts or accessories thereto of either.

In a particular embodiment, the device can include an interface with at least one sensor and drug dispenser or nano-particles not located within the oral cavity.

The systems and devices described as part of the OSWT-OMDDS as laid out in this application can be applied to obtain extensive drug dispenser data and analysis, providing much needed information and assistance, as detailed herein. In particular, a method for obtaining drug dispenser data from an animal or human is provided. The method includes the steps of locating or inserting a device configured to be inserted and securely attached in an oral cavity of an animal or human. The device includes OMDDS receptacle, the receptacle being configured to serve one or more functions within the animal or human's oral cavity without being swallowed. In the method, the smart receptacle is inserted already containing, or, alternatively, receives one or more drug dispensers after insertion, capable of providing or receiving information or analysis relevant to the animal or human. The method includes activating and/or monitoring the one or more drug dispensers, and transmitting or receiving at least some portion of the information or analysis to, from or among a network or networks capable of utilizing the information or analysis.

In the above-described method, the one or more functions of the receptacle can include or consist of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information, or providing a function related to therapeutics, health analytics, diagnostic analytics, performance analytics; integration of body sensors and drug dispensers, health-devices, nano-particles, sports and performance sensors on inanimate objects and sports equipment; customizable developers' tool kit for biosensors, sensors and drug dispensers, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, or providing functions with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors and drug dispensers in order to produce computer-generated precise movement or greater precision and analytics.

In a particular aspect of the above method, at least one of the one or more functions of the receptacle includes or is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function for infants, children, elderly, and animals, providing sleep-aid and preventing snoring and obstructive sleep apnea function, therapeutic, providing a recreational, athletic performance, or sports function, physiological, natural and/or grinding protective function, horse-rider navigation and communication function, vital sign measuring function, diagnostic and therapeutic function, and surgical equipment and hospitalization equipment function.

In various embodiments of the method, information can be transmitted securely to a plurality of remote devices monitoring the animal or human, or information can be transmitted securely to a plurality of remote devices monitoring a plurality of animals or humans.

The OMDDS of the invention combines and provides for communication systems and alerting technology that links a multitude of non-oral or oral saliva-based diagnostic, therapeutic, and medication administration information inputs together. This method of integration of medication administration from oral devices and oral or non-oral diagnostics and therapeutic provide the basis for a real-time or near-time or short or long-term snapshot of effects of medication on an animal or human's health and well-being. Furthermore, these integrate disease symptoms and OMDDS in most beneficial ways for proper health of human and animal individuals.

Accordingly, an OMDDS and administration system, diagnostic oral or non-oral sensors and drug dispenser alerts and communication system, methods, and devices related to and used in conjunction therewith are provided which address the needs and provide the advantages outlined herein.

In an embodiment of the invention, a method is provided for placing in the receptacle the OMDDS, OSWT-OMDDS, In-situ Saliva Component Collection, Concentration, Isolation, and Storage Communication System (ISCCCISCS), In-situ Saliva Component Collection, Concentration, Isolation, and Storage Reader System (ISCCCISRS), In-situ Blood Component Collection, Concentration, Isolation, and Storage Communication System (IBCCCISCS), Insitu Blood Component Collection, Concentration, Isolation, and Storage Reader System (IBCCCISRS), or a combination thereof.

In another embodiment, a method of using transdermal extractions techniques for IBCCCISCS or IBCCCISRS including, but not limited to, electrical, thermal, vibrational or ultrasound, mechanically, chemical enhancers, and electrochemical gradient assisted devices. Examples of electrically assisted are electroporation, ionophoresis, and reverse iontophoresis of thermally assisted are a laser, radio frequency heating, chemical heating, and mechanical heating. Ultrasound-assisted are sonophoresis and phonophoresis; mechanical assisted are micro-needles and tape-stripping; chemical and electrochemical gradient based are salt, natural, human-made polymers and compound assisted diffusion, surfactants, enzymes, salts, chemical- or enzyme-peels, nanoparticles, polymer-chain, microparticulate, etc.

In an embodiment of the invention, a method is provided for networking the OMDDS, OSWT-OMDDS, In-situ Saliva Component Collection, Concentration, Isolation, and Storage Communication System (ISCCCISCS), In-situ Saliva Component Collection, Concentration, Isolation, and Storage Reader System (ISCCCISRS), In-situ Blood Component Collection, Concentration, Isolation, and Storage Communication System (IBCCCISCS), Insitu Blood Component Collection, Concentration, Isolation, and Storage Reader System (IBCCCISRS), or a combination thereof. The method further involves activating or monitoring the one or more sensors within ISCCCISC, ISCCCISRS, IBCCCISC, IBCCCISRS, and drug dispensers to obtain or analyze the information relevant to the health or a physiological characteristic of the human or animal and transmitting at least some portion of the health or physiological information or analysis to a network capable of utilizing the information obtained.

The network capable of utilizing the information obtained from the one or more sensors and drug dispensers can include or consist of one or more network units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, comparative, historical or previous from same or different individual or team, visualizing or presentation development platforms.

The method can include network units which utilize preset ranges, dynamic preset ranges, or degrees of alerts from preset ranges for medical or performance analysis. Additionally, the described method can include network units which utilize biosensors and drug dispenser or sensors measurements for pre-integration and post-integration analyses, as known by those skilled in such arts.

In another embodiment, a therapeutic, diagnostic or other system is provided which includes a device configured to be inserted and securely attached in an oral cavity of an animal or human, wherein the device includes a drug dispenser receptacle. The receptacle is configured to serve one or more functions within the animal or human's oral cavity without being swallowed, and the smart receptacle is configured to contain or receive drug dispensers capable of providing or receiving information or analysis relevant to the animal or human. The system further comprises a smart auxiliary device which is wearable, attachable or insertable externally to the oral cavity. The auxiliary device is configured to obtain information from, provide information to, or both, the one or more sensors and drug dispensers or other medical devices contained in, on, or received by the oral cavity receptacle. The one or more oral cavity sensors and drug dispensers or the auxiliary device, or both, are configured to transmit or receive the information or analysis to or from a network or networks. This system can include an auxiliary device which is also configured to contain or receive one or more drug dispensers and sensors.

The one or more functions can include or be selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function for infants, children, elderly, and animals, providing sleep-aid and preventing snoring and obstructive sleep apnea function, therapeutic, providing a recreational, athletic performance, or sports function, physiological, natural and/or grinding protective function, horse-rider navigation and communication function, vital sign measuring function, diagnostic and therapeutic function, and surgical equipment and hospitalization equipment function or providing a function related to therapeutic and health analytics, diagnostic analytics, performance analytics; integration of body sensors and drug dispensers, medical devices, health-devices, nano-particles, and sports and performance sensors on inanimate objects and sports equipment; customizable developers' tool kit for oral mucosa drug dispensers, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors and drug dispensers in order to produce computer-generated precise movement or greater precision and analytics.

In certain embodiments, at least one of the one or more functions is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function for infants, children, elderly, and animals, providing sleep-aid and preventing snoring and obstructive sleep apnea function, therapeutic, providing a recreational, athletic performance, or sports function, physiological, natural and/or grinding protective function, horse-rider navigation and communication function, vital sign measuring function, diagnostic and therapeutic function, and surgical equipment and hospitalization equipment function.

The system can be set up for use with an individual to obtain information from the individual and transmit it or analysis derived from it directly or indirectly to a network.

The system may utilize a network configured to analyze one or more performance parameters of a group activity or team sport. It may also work with a network configured to analyze one or more therapeutic and athletic performance parameters of humans and animals. Additionally, in an additional embodiment, the system can include a network configured to provide electronic medical records functionality.

The system as described above can include full server access and the system can be configured to analyze individual, group, in-clinic, hospital, or team sports performance as it relates to various body components and drug dispensers.

In one option of the system, the system includes one or more of a digital storage device or full connectivity capability configured to analyze individual patient in home, in clinic, and in hospital therapeutics as it relates to various body component sensors, drug dispensers, or medical devices for real time or historic analysis and review.

As developed by applicants, in certain embodiments the invention is provided as a customizable tool kit or platform for building a therapeutic, diagnostic or other system to provide information, analysis or alerts for an animal, animals, human or humans. The kit or platform of customizable components serves to meet the needs of a developer, consumer or user of variations of the OSWT-oral mucosa drug delivery system. The components include at least one drug dispenser insertable within or upon an oral cavity receptacle configured for placement in the oral cavity of the animal, animals, human or humans, at least one oral cavity receptacle configured to contain or receive the sensors, optionally, at least one smart auxiliary device which is wearable, attachable, insertable, or located on innate objects such as hospital equipment, externally to the oral cavity, and at least one network unit configured to receive information, analysis or alerts from or transmit information, analysis or alerts to, the at least one oral cavity drug dispenser or oral cavity sensors, the at least one auxiliary device, or both, and analyze, transmit, or both, the information, analysis, historic data, or alerts obtained or received. The components for selecting the auxiliary devices, the oral cavity drug dispenser receptacles, the sensors, and the network units are made available to the developer, consumer or user to construct or have constructed an OSWT-oral mucosal drug delivery system configured to obtain or transmit information, analysis or alerts customized to meet their specific needs.

In an embodiment of the OSWT-drug delivery system, the tool kit or platform comes in a variable grouping of preselected sets of kit or platform components or modules of components for constructing the OSWT-oral drug delivery system using the kit or platform, and may come together with instructions for building the desired system. And yet further, in certain embodiments, at least one smart auxiliary component is present in the tool kit or platform.

### DETAILED DRAWING DESCRIPTIONS

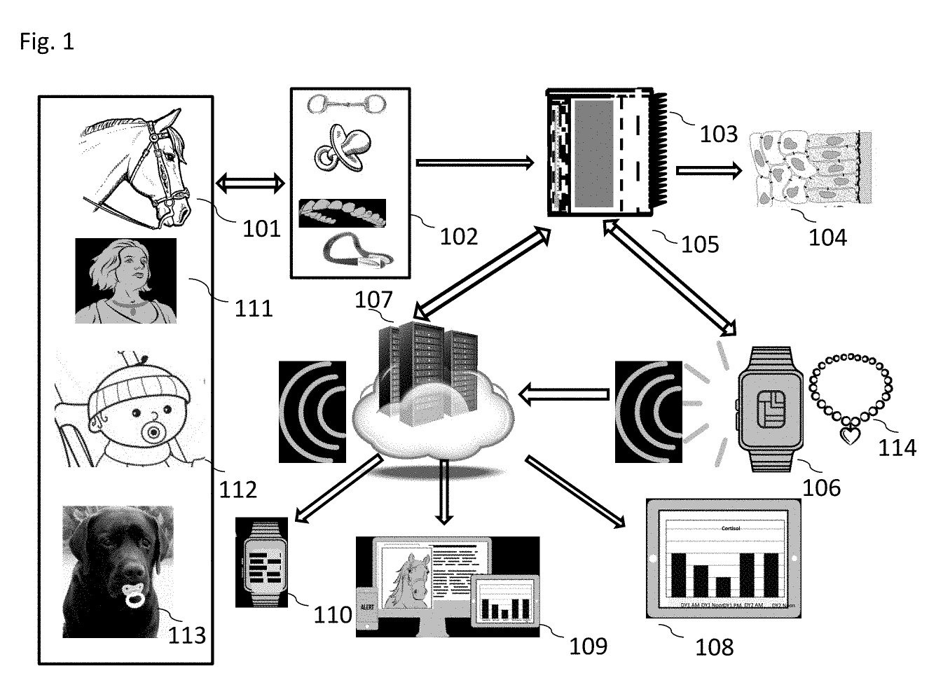

FIG. 1 is a schematic depiction which exemplifies an OSWT-oral mucosal drug delivery system secure wireless communication capability and information retrieval through the animal's or human's oral cavity according to the invention. In these embodiments of the described OSWT-OMDDS, a human, here exemplified by a woman 111, can have OSWT-OMDDS device attached to her teeth through a retainer, or another dental devices such as infant or animal pacifier, equine-bit or equine cheek or lip clip, 101, 111, 112, 113. One or more drugs of various molecular sizes and other physical characters or different formulations can be delivered by OMDDS attached or be part of by any means to the retainer, infant or animal pacifier, equine-bit or equine cheek or lip clip, etc. 102. The OMDDS device with enhancer can make contact with oral mucosa 103, 104, 105. Information can be transmitted via the Internet of things where internet communication chips with their own IP address and provides the ability to transfer data over a network contained in the OSWT-OMDDS in oral cavity device 105. In some embodiments, information gathered could be stored in OSWT-OMDDS 105. The smartwatch, necklace, other accessories, or wearables 106, 114 transmits the oral cavity information to one or more remote PC laptops, tablets, smart-phones, wearable, or reader communication devices through the owner's WiFi in accordance with embodiments of the present invention 108, 109, 110. The information can be transmitted, e.g., through an internet of things, RFID tag, blue tooth 5, or other communication methods on an accessory, smart jewelry, clothing, watch, other accessories, on, in, or around the woman, exemplified here by a smartwatch 106 or smart necklace 114. The OSWT-OMDDS can include a Wifi reader, RFID tag reader, blue tooth chip, or etc. placed within or in proximity to any part of the oral cavity, temporarily or permanently. The signal is then transmitted to a secure server or cloud 107. Information could be stored or sent from the server or cloud to several different smartphones, smartwatches, computers, and others. The alert, if needed, can be dispatched to any computer-aided device or emergency dispatch if the OSWT-OMDDS detects higher than average or abnormal metabolic ranges, for example. This communication system can also be used to control drug delivery through this system.

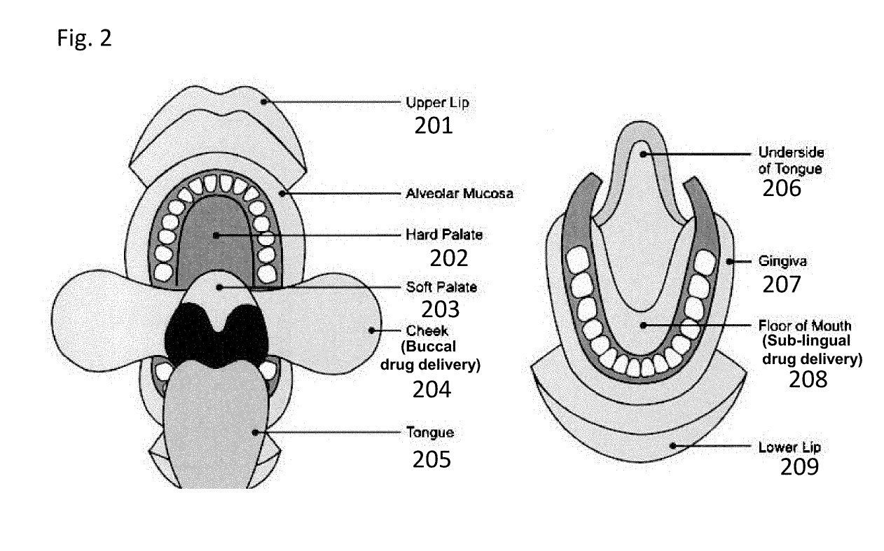

FIG. 2. Oral Cavity For Drug Delivery: The oral cavity consists of the upper and lower lips 201 and 209, cheek 204, tongue 205, hard palate 202, soft palate 203, gingiva 206, and floor of the mouth 208. The lining of the oral cavity is called the oral mucosa, and includes the buccal, sublingual, gingival, palatal and labial mucosa. Drugs can be delivered through cheek (buccal) mucosa 204 and through the floor of the mouth (sublingual) mucosa 208. These regions are attractive sites for drug delivery due to the presence of non-keratinized epithelia so are more permeable. Buccal drug delivery can be used for mucosal (local effect) and oral mucosa (systemic effect) drug administration 204 and 208. For the local drug delivery, the aim is to achieve the site-specific release of the drug; whereas, for the systemic, drug absorption through the mucosal barrier to reach the systemic circulation is required. Sublingual delivery is generally systemic delivery of drugs through the mucosal membranes lining the floor of the mouth 208. Anyone familiar with the arts understands that because cheek (buccal) mucosa and through floor of the mouth (sublingual) mucosa have non-keratinized cells and form lesser of the barrier, they are better suited for drug delivery with or without enhancement systems. Other parts of the oral cavity can also be used for drug delivery, but due to the presence of keratinized cells, they might be less efficient. Still, due to the abundant blood supply and with use enhancing techniques might make drug delivery efficient through oral mucosa as compared to the skin.

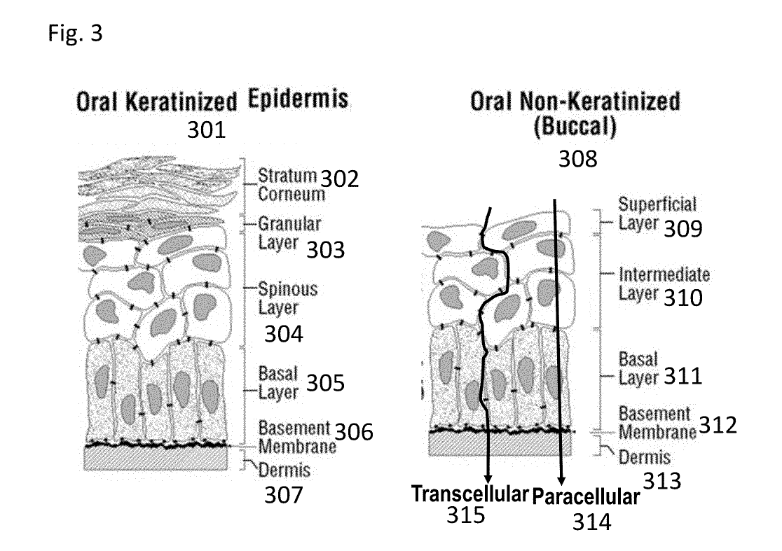

FIG. 3. Cellular Structure of Oral Mucosa: The oral mucosa is of two types, keratinized 301 and non-keratinized 308. The mucosa of areas that are subjected to mechanical stress such as the gingiva and hard palate is keratinized and is similar to the epidermis of the skin 301. However, the mucosa of the soft palate, sublingual, and buccal regions are not keratinized and formless barrier for drug delivery 308. The keratinized epithelia contain neutral lipids like ceramides and acylceramides are associated with the barrier function 302. These epithelia are relatively impermeable to water. In contrast, non-keratinized epithelia, such as the floor of the mouth and the buccal epithelia do not contain acylceramides and only have small amounts of ceramides. They also contain small amounts of neutral but polar lipids, mainly cholesterol sulfate and glucosylceramides. These mucosa have been found to be considerably more permeable to water than keratinized epithelia 308. Beneath the epithelium is the basement membrane, lamina propia and submucosa 304, 305, 309, 310, and 311. The oral mucosa also contains many sensory receptors including the taste receptors on the tongue. Drugs can be transported across epithelial membranes by several pathways such as passive diffusion, carrier-mediated, active transport or other specialized mechanisms. Buccal absorption is predominantly passive diffusion across lipid membranes via either the paracellular 314 or transcellular 315 pathways. The hydrophilic nature of the paracellular spaces provides a permeability barrier to lipophilic drugs but is favorable for hydrophilic drugs. In contrast, the transcellular pathway involves drugs penetrating through the cells before the drugs enter the systemic circulation. The lipophilic cell membrane offers a preferable route for lipophilic drugs compared to hydrophilic compounds. Drugs can transverse both these pathways simultaneously or a particular route could be preferred depending on the physicochemical properties of the drug molecules. Saliva provides a water-rich environment for drug release from their delivery systems especially those based on hydrophilic polymers. However, saliva flow decides the time span or the residency time for the released drug at the delivery site. This flow can lead to premature swallowing of the drug before effective absorption occurs through the oral mucosa and is referred to as “saliva wash out.” However, the invention presented here can prevent “saliva wash out” by modulating the residency time or retention time, increasing the contact area, optimizing contact angle, and/or etc. of the drug in the oral cavity in a more controlled manner and limiting exposure to the saliva. Because the rate of blood flow through the oral mucosa is substantial, it is not the rate-limiting factor in the absorption of drugs through the oral cavity.

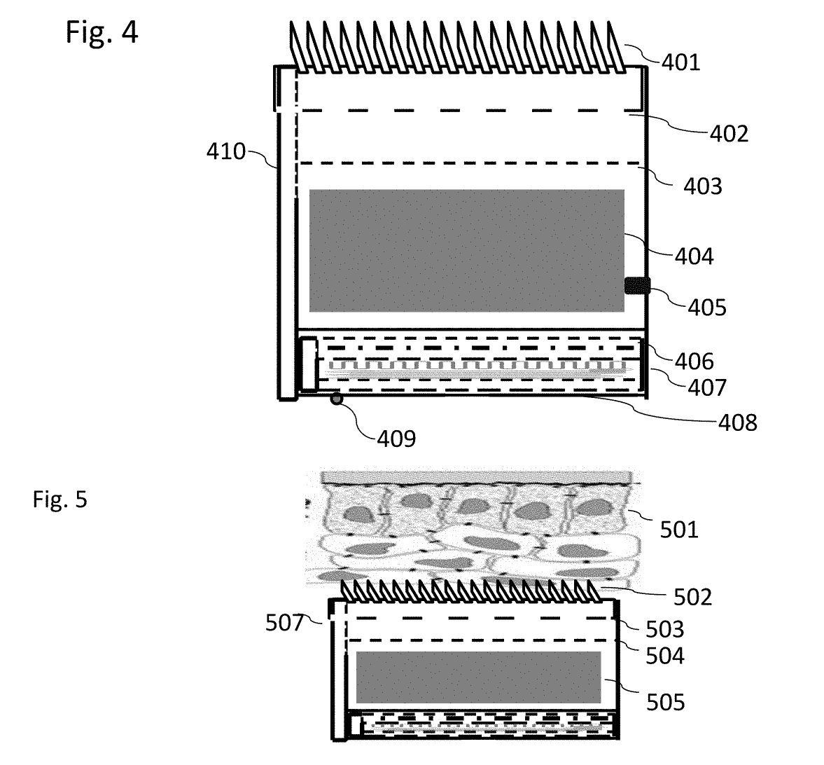

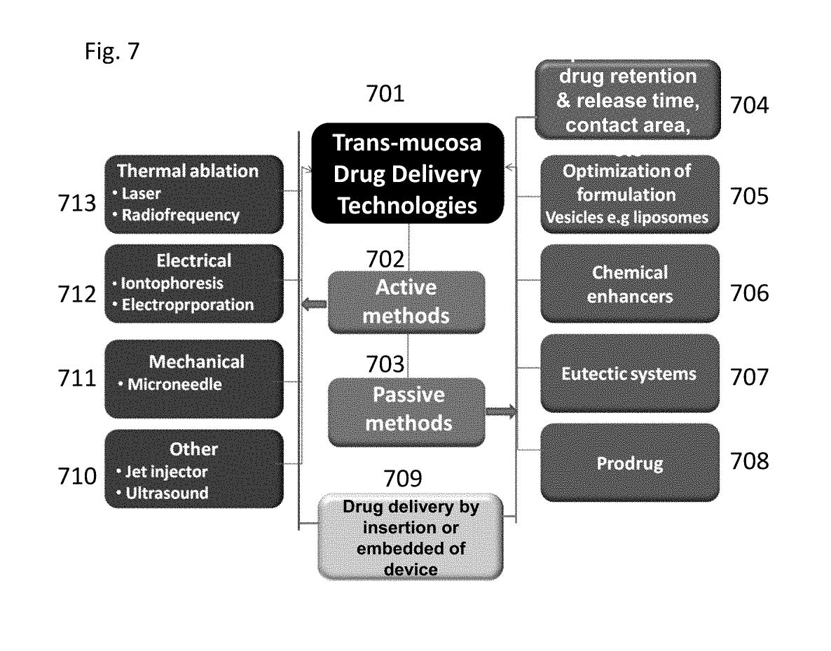

FIG. 4 depicts a schematics depiction that exemplifies the structure of an oral mucosa drug delivery device along with the enhancers and biosensors, forming OMDDS, as may be used in embodiments of the invention. The drug delivery enhancing part of the device can be in contact with mucosa lining 401. However, when the enhancing part of the OMDDS is absent, the OMDDS might be in contact with mucosa or might not be in contact with the mucosa and face oral cavity. The enhancer of OMDDS device can utilize one or more passive or active technologies described in detail in FIG. 7. A permeable membrane is present 402 and would be in direct contact with the mucosa in the absence of the enhancer. Since the permeable membrane allows all drug molecules to be released, so another membrane, control membrane, can control the amount and type of molecules within the drug formulation 403. It is understood by anyone familiar with the arts that this device could contain preservatives and other chemicals routinely used to enhance OMDDS and storage of the drug. The drug is stored in a reservoir 404, which might be refilled through 405; in another embodiment, the drug reservoir can be replaced or the entire OMDDS module can be replaced so these devices can be repeatedly used. The OMDDS device could contain software for the device functionality, one or more electrical input; one or more transducers configured to convert input electrical energy to different energy forms for activating and implementing extraction procedure; a controller configured for providing the control signals, intensity, duration, timing, sequence, and nature information for the different energies supplied to the device said transducers, etc. 406, 410. The drug delivery could also be controlled by use of a pump or an infusion pump 411. It is understood by anyone familiar with the arts that the organization modules or device parts such as OMDDS device, a software module, transducers, controller, collection and concentration chips, etc. presented here could be organized in a variety of manners depending upon ease of use, manufacturing feasibility, regulations, etc. These membranes are non-permeable or impermeable to ensure that drug molecules do not leach to the other via the side or backing membranes, 407, 408, and 410. The device can also contain sensors 409. Furthermore, the device could also contain or be coated with pain-killers or pain-numbing agents for patient comfort. These devices could be custom-made by 3D printing.

FIG. 5 depicts a schematics depiction that exemplifies OMDDS in contact with oral mucosa, as may be used in embodiments of the invention. The mucosa of areas that are subjected to mechanical stress such as the gingiva and hard palate is keratinized and is similar to the epidermis of the skin 501. However, the mucosa of the soft palate, sublingual, and buccal regions are not keratinized and formless barrier for drug delivery 502. The drug delivery enhancing part of the device can be in contact with mucosa lining 508. However, when the enhancing part of the OMDDS is absent, the OMDDS might be in contact with mucosa or might not be in contact with the mucosa and face oral cavity 509. Both drug delivery device with or without the enhancing part could be flexible, non-flexible, moldable, contain micro- or nano-hinges, -pivots, -hinges with micro- or nano-teeth, dial, others, or a combination thereof 508, 509. This design could ensure proper and efficient contact between the device and the oral tissue. Furthermore, the contact between some or all parts of the device that deliver drugs could be customized via flexible, in-flexible, mouldable, micro- or nano-hinges, -pivots, -hinges with micro- or nano-teeth, dial, others, or a combination thereof to each individual for best drug delivery. These could also assist in manual or automatic control of most efficient the oral tissue contact area, contact angle, contact time, etc., 508, 509. Since the permeable membrane allows all drug molecules to be released, so another membrane, control membrane, can control the amount and type of molecules within the drug formulation 503. It is understood by anyone familiar with the arts that this device could contain preservatives and other chemicals routinely used to enhance OMDDS and storage of the drug. The drug is stored in a reservoir 504, which might be refilled through 405; in another embodiment, the drug reservoir can be replaced or the entire OMDDS module can be replaced so these devices can be repeatedly used. The OMDDS device could contain software for the device functionality, one or more electrical input; one or more transducers configured to convert input electrical energy to different energy forms for activating and implementing extraction procedure; a controller configured for providing the control signals, intensity, duration, timing, sequence, and nature information for the different energies supplied to the device said transducers, etc. 506, 507. It is understood by anyone familiar with the arts that the organization modules or device parts such as OMDDS device, a software module, transducers, controller, collection and concentration chips, etc. presented here could be organized in a variety of manners depending upon ease of use, manufacturing feasibility, regulations, etc.

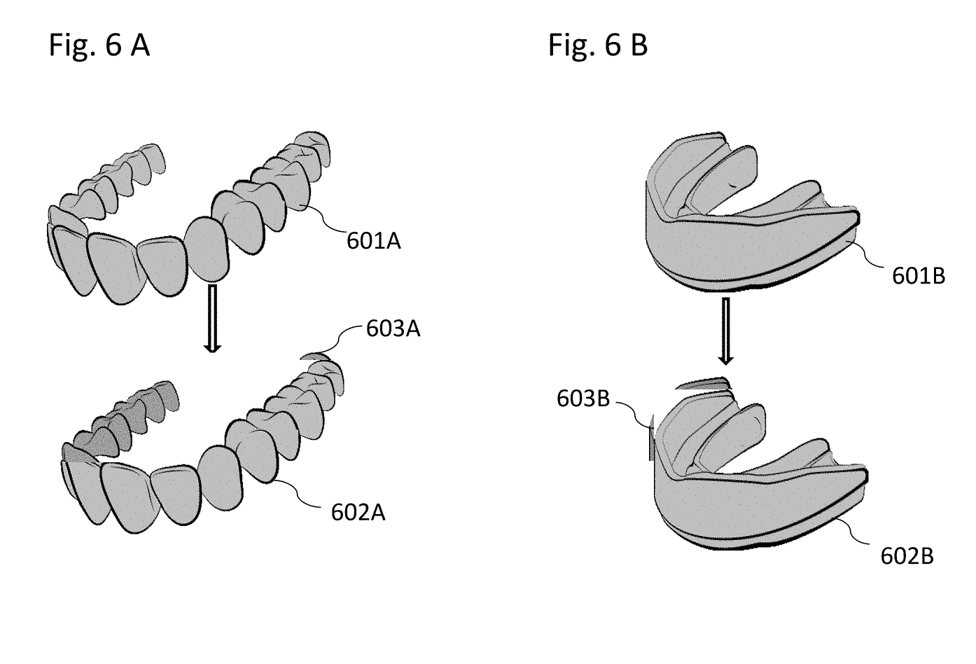

FIG. 6 depicts embodiments which exemplify the locations of OMDDS on a retainer and a mouth guard.

FIG. 6A depicts embodiments which exemplify the locations of OMDDS on a retainer. The placement of OMDDS on retainer or any other dental device would be determined by several factors including, but not limited to, anatomy and cellular structure of oral tissue, location of the salivary glands, physical and chemical properties of drug to be delivered, drug formulation and concentrate, and production and manufacturing requirements and ease, patient comfort and convenience, and regulations. It is understood by anyone familiar with the arts that more than one embodiment are possible. However, depicted herein is an embodiment where the OMDDS is located on the inner side of lower jaw retainer but away from gland opening submandibular and sublingual duct 603A. Whereas, 601A, 602A depicts front of the retainer, which is unchanged. This location is away from submandibular and sublingual duct openings, which would cause minimum drug was-out effect and drug ingestion, yet in contact with sublingual mucosa, which is best for drug delivery in the oral cavity. The OMDDS could be permanently or temporarily attached to the retainer, and the temporary attachment could be detachable such that the user or caretaker could attach or de-attach it. All can be refillable.

FIG. 6B depicts embodiments which exemplify the locations of OMDDS on a mouth guard. The placement of OMDDS on the mouth guard or any other dental device would be determined by several factors including, but not limited to, anatomy and and cellular structure of oral tissue, location of the salivary glands, physical and chemical properties of drug to be delivered, drug formulation and concentrate, and production and manufacturing requirements and ease, patient comfort and convenience, and regulations. It is understood by anyone familiar with the arts that several embodiments are possible. However, depicted herein is an embodiment where the OMDDS is located on the outer side of upper jaw mouth guard 603C. Whereas, 601C, 602C depicts front of the mouth guard, which is unchanged. This location is cheek mucosa, which would assist in fast drug delivery through cheek mucosa. For drug delivery for local infections, for example, the OMDDS can be facing towards inside of the mouth guard and in contact with the infected area, provided the infected are is upper gums. The ISCCCS could be permanently or temporarily attached to the mouth guard, and the temporary attachment could be detachable such that the user or caretaker could attach or de-attach it. All can be refillable with the drug for more than one repeat use.

FIG. 7. is a schematic representation of various technologies to enhance drug delivery through the oral cavity: The drug delivery can be enhanced either by passive or by active methodologies or both 702, 703, and 709. Passive methods predominately include increasing the drug retention time, optimum drug contact area and angle, controlled drug release, anchoring of the drug, the influencing of drug and vehicle interactions and optimization of formulation, and others 704, 705, 706, 707, 708, and 709. Passive methods are relatively easy to incorporate into trans-mucosal devices such as patches and dental devices through chemical enhancers and emulsions 705, 706, and 707. One of the most widely used passive approaches is the use of chemical penetration enhancers which facilitate drug permeation across the mucosa by increasing drug partitioning into the barrier domain of the keratinized or non-keratinized layers. Penetration enhancers have several mechanisms of action such as by increasing the fluidity of the lipid bilayers, interaction with intercellular proteins, disruption or extraction of intercellular lipids, increase of the drug's thermodynamic activity and increase in mucosa hydration 705, 706, 707, and 708. Penetration enhancers can be divided into several groups based on their chemical structure, and most have mixed modes of action, some common examples investigated penetration enhancers are alcohols, sulphoxides, azone, pyrrolidones, essential oil, terpenes and terpenoids, fatty acids, water, and urea. However, the major limitation for penetration enhancers is that their efficacy is often closely correlated with the occurrence of tissue irritation. Variations of semisolid gel vehicles such as proniosomes and microemulsion gels are used as penetration enhancers. Proniosomes are non-ionic based surfactant vesicles and are also known as “dry niosomes” because they may require hydration before drug release and permeation through the mucosa. Proniosomal gels are used because they act as penetration enhancers that enhance the drug permeation from the mucosal barrier. When hydrated proniosomes are converted into niosomes which are capable of diffusing across the stratum corneum and adhere to the cell surface which causes a high thermodynamic activity gradient of the drug at the vesicle/mucosal surface, thus acting as the driving force for the penetration of lipophilic drugs across the mucosal 708. Thus niosomes can act as a penetration enhancer for drug molecules. Some of the limitations associated with penetration enhancers are poor efficacy, safety, and local irritation and inflammation. Penetration enhancers might not achieve the desired skin disruption and their ability to increase transport across the skin is low and variable. Penetration enhancers can potentially cause skin irritation including local inflammation, erythema, swelling and dermatitis. Still, one main drawback of passive methods can be lag time in drug release that can have several negative effects when rapid drug delivery is required such as insulin. These drawbacks are predominately due to keratinized epidermis barrier of the skin and can be similar in keratinized gingiva and hard palate. However, the mucosa of the soft palate, sublingual, and buccal regions are not keratinized and are lesser barrier for drug delivery, and thus, are better for drug delivery, safer, and without any or diminished local mucosa irritation. Because of faster healing properties of oral mucosa, and mucosal irritation would be quickly healed. Our devices will also enhance and better control the role of saliva in mucoadhesion for drug delivery systems which work on the principle of adhesion to the mucosal membrane with drug molecules, and thus extend the dosage form retention time at the delivery site 704. Our devices can use the eutectic system to enhance retention time through a decrease in formulation viscosity 707. Conversion of pro-drugs as part of our device to drugs in the oral cavity can also enhance drug delivery system 708. Current, limitations associated with oral drug delivery include uncontrolled swallowing of the released drug into GI tract and difficulties in holding the dosage form at the site of absorption. These disadvantages can also be overcome by our device. Our device can increase retention time, area and angle to enhance drug absorption in the buccal and sublingual cavities 704. The dissolution or disintegration of lozenges is usually controlled by the patient by time and force of suck the unit. Our device would offer better control over of the drug dissolution or disintegration of the drug units 704. Therefore, our device can decrease inter- and intra-individual variations in absorption and bioavailability of drugs delivery. In addition, our device provides low cost, easily administration drug delivery system that will have high patient compliance.

Our device can be used with active methods for mucosal permeabilisation include ultrasound, electrically assisted methods (electroporation and iontophoresis), velocity based devices (powder injection, jet injectors), thermal approaches (lasers and radio-frequency heating) and mechanical methodologies such as microneedles (MN) and tape stripping 709, 710, 711, 712, and 713. These approaches allow a broader class of drugs to be delivered into the tissue. Active methods involve the use of external energy to act as a driving force for drug transport across the mucosa or by physically disrupting the keratinized or non-keratinized mucosa; the energy could be used to break the barrier as in insertion, embedded, microneedles, jet injectors 709, 710, and 711. The energy could be provided either by a power source 713, 712 or by mechanically by the user or installer if different than the user 709, 710, and 711. These techniques greatly expand the range of drugs that can be delivered effectively across the tissue. In addition, active methods also offer more reproducible control over the delivery profiles of the medications, thus overcoming lag times between the application and the drug reaching the systemic circulation when compared to passive methods. Some adhesive systems deliver the drug towards the mucosa only with an impermeable product surface exposed to the oral cavity which prevents the drug release into the oral cavity, so our device exposure areas on the inside or outside or both sides of the device. The drug in our device can be liquid, semi-solid, solid, aerosol, sprays, prescription, non-prescription, herbal, and others.

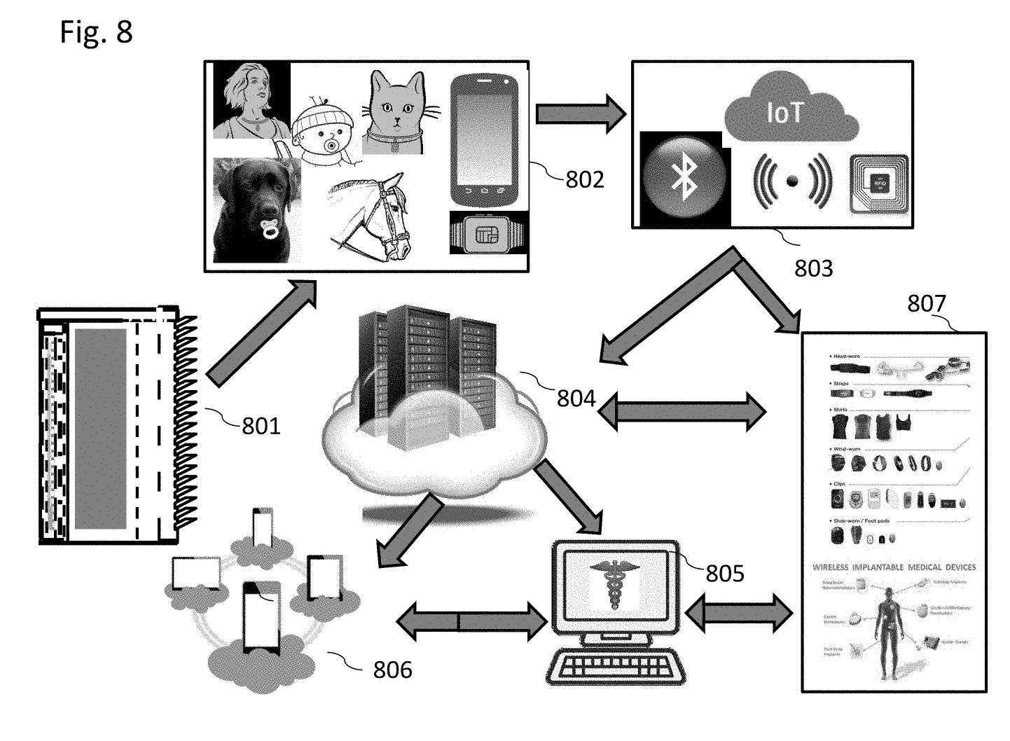

FIG. 8. represents OSWT-OMDDS network of drug delivery with diagnostic and biometric analysis. In these embodiments of the described drug delivery system, an example of a fully integrated OMDDS, can be standardized or customized and provided as a customizable tool kit for humans, including an infant's or animal's smart pacifier, smart horse-bit, etc. and integrated through communicating devices, e.g., smart cat collar, smartwatch, smartphone is depicted 801 and 802. These smart devices can communicate via blue tooth, Wifi, internet of things, etc. 803. In 804, advanced computer processing or secure server is indicated which can evaluate one or more variables originating from an individual (or animal). 807 wearables worn on the body, 806 and 805 input from all OMDDS, medical devices and other sources (medical diagnostic, medical wearables, medical devices, temperature, accelerometer, gyroscope, inertia-sensors and drug dispenser, sensors, etc.), 807 input from all media, wearbles, classifications (audio, visual, touch, olfactory, taste, ophthalmoception, audioception, gustaoception, olfacoception or olfacception, tactioception, (thermoception), kinesthetic sense (proprioception), pain (nociception), balance (equilibrioception), vibration (mechanoreception), and various internal stimuli (e.g. the different chemoreceptors), tension sensors, pressure, stretch receptors, thirst, hunger, and time perception etc.), and 807 environment sensors, body sensors and wearables, drug delivery system, etc. The integration and amalgamation of the aforementioned can comprehensively 805, 806, and 807. The drug delivery system collectively provides the users and health care providers secure bi-directional communications, comparatives, historical analysis, time-stamped data, reporting, and feedback. Also, the drug delivery system can be synced with all wearables and other biosensors and other drug dispensers as shown in 807.

The oral cavity is a semi-sterile, clean, fast wound healing environment, and has a high threshold for pain because oral secretions include antiseptic-like molecules, wound healing, and pain-killer biologics which are unique to this cavity. In animals, these properties are apparent when they groom themselves by using their tongue to lick away dirt and pathogens, yet no infection results from the licking of pathogen-laden dirt. Therefore, oral mucosa drug delivery enhancers of this invention can be more comfortable and painless used as compared to transdermal drug delivery via the skin.

Because animals are unable to communicate about their health, an OSWT-oral mucosal drug delivery system, as described, becomes an important part of their therapeutics and healthcare management. The herein described OSWT-oral mucosal drug delivery system permits animals and pets to benefit from these drug dispensers and biosensors because their health status can be monitored and intervention provided in real time and preemptively to even save their lives and help in their propagation.

Unlike any other body cavities, the oral cavity represents a unique cavity because it is easily accessible, is highly vascularized, has a very absorptive surface, and secretes several biologics. In addition, salivary secretions also contain fast healing and analgesic properties. These properties are experienced by most people during their daily teeth cleaning routine that often causes painless and fast healing gum bleeding. This example demonstrates the naturally-occurring aseptic, pathogen-destroying, and natural healing properties of saliva. Compared to currently used body parts, such as skin (which in animals adds extra complications due to the presence of fur) used to place or implant drug dispensers, OMDDS are less painful, heal fast and are more comfortable since any broken tissue during drug dispenser placement, insertion, or use of drug enhancers is fast healing, has less of a possibility of infection. These qualities make oral mucosa drug dispensers and other oral sensors less invasive or minimally invasive. Discrete without being visible and yet easily accessible, they can be worn round the clock, including during rest, sleep, high activity, and more. Additionally, they are readily calibrated, rejuvenated, drug replaced, and/or cleaned. An increase in a patient, both human and animal, compliance would result from their non-invasive or minimally invasive nature and ease of accessibility. Further, a prolonged lifespan of these drug dispensers is possible due to their protection from external elements and placement in a relatively aseptic environment. Thus, these oral mucosa drug dispensers, used in accordance with the invention, increase drug delivery efficiency, reduce healthcare cost while simultaneously increasing patient compliance. Oral mucosa drug dispensers used as described herein are superior to other drug dispensers in that they can deliver drugs intermittently, frequently, or constantly as needed to treat without drug damaged in the gut or without the use of invasive methods and manage most diseases. Once the oral mucosa drug dispenser is placed in the oral cavity, it can monitor and collect data for short or long term.

Drug delivery through the oral cavity is capable of augmenting non-invasive medication bio-availability, therapeutics, and drug delivery. However, currently available methods for the oral medication administration are inefficient and do not alert or communicate information about medication administration. Further, current usage of the oral cavity for drug delivery or medication administration is immobile and uses the rudimentary method for the administered drug by unattached placement of the medication and similar means. Thus, there exists a critical need for improved devices, methods and systems in animal and human oral cavity drug delivery system, and integration of biosensors and drug dispenser technology and its use for physiological and health information gathering, assessment, monitoring, and ultimately, health care and therapeutic assistance with oral drug administration and delivery. Moreover, a major side effect of these medication delivery techniques avoids cell injury and death as is the case of skin, but such injury can be easily and quickly healed in the oral cavity tissue.

Also, currently there is a profound lack of integration between a multitude of cross-linked technologies and skills when determining information regarding metadata diagnosis; with geometric tracking, multimedia, communication networks, analytics, alerting, and kinematics for individuals, team sports, organizational groups, animals and humans, which enhance health and performance. In addition, these current limitations restrict a multi-dimensional approach which could seamlessly measure individuals and animals with greater accuracy, convenience, yet far less intrusively. In addition the lack of integration between disciplines fails to address the growing need for the next level of metadata and biological tools which could provide early detection of diseases and medical disorders with greater accuracy as stated herein in the present invention.

Cheaper and personalized drug dispensers and/or sensors of the invention become important in managing healthcare costs and outcomes for multiple diseases. As technologies such as nano-technology or 3D printing become more advanced and easily and cheaply available, the OSWT-oral mucosal drug delivery system of the invention may be adapted to provide additional savings in healthcare costs for both humans and animals. Furthermore, the present invention enables drug dispensers and/or sensors to be uniquely communicated from one or more animals and humans to a plurality of OSWT-oral mucosal drug delivery systems; thus, adding to the utility of these drug dispensers. The OSWT-oral mucosal drug delivery system of the invention enables humans to monitor and alert themselves or others and enables animal-OSWT-oral mucosa drug delivery units to send alerts to humans to monitor and alert their caretakers. The present invention systematically integrates all forms of the oral-based sensors and drug dispensers in order to offer new, innovative, and unique opportunities to monitor the overall health status of both humans and animals. They also help to preemptively and accurately diagnose, detect, and monitor, and thus, help in the prevention, prognosis, and risk-assessment of a variety of diseases. The oral cavity is a window into the overall health, and disease status for the entire body and the OSWT-oral mucosal drug delivery system of the invention enables and integrates a multitude of the aforementioned devices to quickly and efficiently communicate a person or animal's health and therapeutic landscape and schematic map.

In some cases, OMDDS can accurately catalog and send data to user's medical records about when and how much drug was delivery. Moreover, since the OMDDS is integrated with sensors and other medical devices, the amount of drug delivered can be correlated with symptoms, vitals, and biometrics measured by other sensors, both wearable and non-wearables. The drug delivered can also be correlated with other medical devices including saline and blood pumps to better inform physicians about total health of an individual in clinic, hospital or at home.

The OSWT-drug delivery system of the invention is uniquely developed to provide highly customizable information and alerts. This is in part due to the large and highly variable number of sensors, and drug dispenser receptacles, accessory receptacles, network interfaces and network components that may be utilized and combined.

Accordingly, in a particularly advantageous set of embodiments, the invention offers an OSWT-drug delivery customizable developers' or development tool kit or platform which provides people, companies, organizations, hospitals, teams, institutes, or others to create their own OSWT-oral mucosal drug delivery system and employ OSWT-oral mucosa drug delivery methods which are custom-tailored to meet their specific needs and optimum circumstances. For example, using the OSWT-oral mucosa drug delivery developers' customizable tool kit or platform, the needs of patients, sports teams, and organizations can utilize a system for one or more settings, ranges, dashboards, historical or other data graphs, biometrics, analyses, and/or for their own market-based. Differing temperature alerts in response to drug delivered by OMDDS may be monitored when a pre-established range is adjusted based on customized datasets. The devices of the OSWT-drug delivery system can be wireless and completely contained in the oral cavity, or may use accessory sensors and drug dispensers (or external ones). The OSWT-oral mucosal drug delivery tool kit and software may be customized by physicians, coaches, etc. to, e.g., amount of drug delivered, monitor vitals, etc. Alerts, as described elsewhere herein can be triggered remotely to read periodically on an as-needed basis, and the data stored on a chip or system, and then streamed, or directly streamed and transmitted.

A feature of many embodiments of the OSWT-oral mucosal drug delivery system and methods of the invention is the dynamic nature of the integrative alert components that may be built into the customizable operations.

3D printing applied to the invention makes cost-effective and less costly custom dental devices available and thus makes our OSWT-oral mucosal drug delivery system more cost-effective. In one embodiment, the present invention involves the pre-set amount of drug delivery which can be as needed either continuous or non-continuous for treatment of a disease. However, the oral mucosa drug dispenser system of the present invention offers a flexible and unique opportunity to deliver drugs in a home setting, at work, during daily activities, or even while sleeping. The device may be present in the oral cavity for a short or long time and may be temporary or semi-permanent. The OSWT-oral mucosal drug delivery system can be applied and may be part of a plurality of dental or oral devices as detailed elsewhere herein. The OSWT-oral mucosal drug delivery system could also be applied to devices that are partially inside the oral cavity and partially outside the oral cavity such as pacifiers, smart thermometers, a smart gauge, smart dipstick, smart rod, smart stick, a smart device to collect blood or saliva, etc. A plurality of drug dispensers, sensors, and RFID, micro- and nano-communication devices, micro- and nano-enabled energy harvesting, micro- and nano-energy storage, micro- and nano-devices or components may be installed in any dental device and/or placed in an oral cavity by means known to one skilled in such arts, depending upon the medical or diagnostic intent.

In yet another embodiment, the OSWT-oral drug delivery systems of the invention may be used in conjunction with other smart wearables or attachable on one or more individuals creating a measurable therapeutics and diagnosis data.

OSWT-oral mucosa drug delivery materials inserted in the oral cavity as described in the present invention may detach, clip, be alerting chips, etc., and can be constructed for a short time period (temporarily) or constructed for a long-term (e.g., semi-permanent) time period. All time periods for the plurality of orally inserted and attached devices used for one or more functions of the invention may be deployed. Oral devices employed include, but are not limited to, drug dispensers, RFID tags, and any inserted attached or detachable dental device used for humans and animals to obtain data and sensors and drug dispenser-derived information. The design and construction of the inserted oral device will vary for each species and is customizable to deliver, monitor and/or alert for all types of medical conditions. Besides, alerts mentioned elsewhere, alerts can be closed loop or open loop; when the amount of drug delivered by OMDDS is automatically changed in accordance with preset values for a biometric or other health and diagnostic parameter and drug amount, this close loop treatment can also be send as an alert. However, when a user or a healthcare provider changes the drug amount delivered by OMDDS in response to changes in diagnostic parameters as determined by biosensor, sensors, medical devices, etc., the alert is open loop. Both of these are very useful in healthcare management of an animal or human individual.

In addition to oral drug dispensers, the OSWT-oral mucosal drug delivery system of the invention includes sensors, biosensors and drug dispensers, and nano-particles located elsewhere within and/or on an animal or human that interfaces with the OSWT-oral mucosal drug delivery system. Motion sensors in a room might be used to determine the gait of a patient. These sensors may also interface with OMDDS to determine the cause of a patient's poor gait as it relates to side-effects or therapeutic value of the delivered drug. Accurate information regarding the cause of the patient's poor gait would help in preventing accidents resulting from his or her poor gait. The OSWT-oral mucosal drug delivery system also can interface with various other devices located on or near an animal or human and securely communicate with a plurality of remote devices monitoring the health, therapeutic, and/or well-being of one or more animals or humans. These devices are optionally within a human or animal body, e.g., such as a drug dispensing pump or other. Alternatively, the device may be located on the human or animal's body, such as a cardiac monitor, dog collar, and accessories or other. These devices may read and interface with a variety of diverse biosensors and drug dispensers such as blood pressure and heart rate components of the OSWT-oral mucosal drug delivery system with a cardiac monitor and a drug dispersing pump as one example. The OSWT-drug delivery system may also communicate with nano-particles in any body part.

In a further embodiment, one or more databases, secure servers, or other devices are utilized to store and/or capture data. The database contains data collected from the OSWT-oral mucosal drug delivery system and any data input in the system including but not limited to data used to send alerts; here, data encompasses both data captured and collected from OSWT-drug delivery and data used to send out alerts. One or more software programs may collect, capture, and store data obtained from any of the OSWT-oral mucosa drug delivery interfaces. Some of this data will be publicly available and viewable, and some of the data will be only for private viewing and available to the relevant users and healthcare providers. To prevent any loss of data, in certain embodiments, when connectivity is lost and data cannot be transmitted in real time to a central storage system, data is temporarily stored on the local device and transmitted to a central system after connectivity is restored. These software programs may be responsible for any and all data related aspects such as, but not limited to, data comparative with historical or previous data of same individual or other, management, characterization, filtering, transformation, sorting, processing, modeling, data mining, queries, browsing, inspecting, investigation, retrieval, integrating, dissemination, qualitative, quantitative, symmetric, asymmetric, normalizing, clustering, correlations, computer derived values and rages, simple or complex mathematical calculations and algorithms, analytics, statistical data, predictive data, integrations, interpretation, exploratory, finding abnormalities, performance, data products, consumer data, server data, visualizing and/or presentation in a variety of platforms. Here, data analysis also means software for that analysis for disease and other diagnosis and analysis for both humans and animals. Thus, this software may supplement or partially and/or fully replace a healthcare provider's input, such as that of a physician, veterinarian, etc., or in a non-medical context. It is understood by anyone familiar with the art that the present invention could lower the cost of health-care, and therefore, insurance companies could cover the cost of these devices for both humans and animals.

Oral mucosa drug dispensers and/or RFID tags can be attached, embedded, glued, inserted, etc. to a pre-existing oral device. The oral drug dispensers can be inserted by themselves into the oral tissue. This drug dispenser, when needed, can be removed by oneself at home. The dental device containing the drug dispensers can be disposable, clip-on, stick-on, adjustable, moldable, flexible, inflexible, attachable, and/or removable.

Listed below are examples of embodiments of this invention; the medical, therapeutic, ranges, and preset values used here are only used as examples. It is understood by anyone familiar with the art that many more examples are possible with adult and babies humans and animals without departing from the scope of the invention. Anyone familiar with the arts knows that the drugs mentioned below can be replaced by any other drug or medication, and depending upon drug molecular size and characteristics, one or more of oral drug delivery enhancement systems along with optimized contact area, contact angle, release time, etc. can be utilized for maximum drug delivery. The OMIDDS device can be chosen from a variety of oral or dental devices for humans and animals mentioned elsewhere and can be used at home, in the clinic, or in a hospital setting.

### EXAMPLE 1

In one embodiment of the inventions, Pamela suffers from Pernicious anemia, so suffers from vitamin B₁₂ deficiency. Systemic pernicious anemia is an auto-immune disease and causes her immune system to attack the cells in her stomach that produce the intrinsic factor, which is essential for vitamin B₁₂ absorption in the gut. She takes sublingual vitamin B₁₂ pills or liquid; however, only a small part of the vitamin is absorbed via oral mucosa, and most B₁₂ is ingested and remain unabsorbed. To avoid B₁₂ deficiency symptoms, Pamela gets monthly B₁₂ shots or injections which are painful, inconvenient, time-consuming, and expensive. The invention detailed herein can remove the need for ingestion. Pamela would wear a dental device such as full or partial retainer, mouth guards, night guard, etc. with vitamin B₁₂ fill able or replaceable receptacle with optimized contact area, contact angle, release time, etc. such that maximum amount of the vitamin is absorbed and minimum is washed away by saliva and is ingested. She wears the device for duration and frequency of delivery of an optimum amount of the vitamin.

Anyone familiar with the arts knows that vitamin B₁₂ can be replaced by any drug or medication, which is not effectively absorbed in the gut.

### EXAMPLE 2

Another example of the utility of this invention is the use of a dental device for treatment of local disease such as gingivitis. Peter suffers from gingivitis; if left untreated with time, the gingivitis plaque can spread and grow below the gum line. The chronic inflammatory response can be caused by bacterial toxins, which cause the body to turns on itself and the tissues and bone that support the teeth are broken down and destroyed. As gums separate from the teeth, the spaces between the teeth and gums become more infected further destroying both bone and gum tissue, which can eventually loosen teeth. To treat such inflammation and infection, Peter can wear a dental device with OMDDS-anti-inflammation and OMDDS-antibiotics respectively. Currently, there are no such technologies available; for treatment of local inflammation, system anti-inflammatory drugs are used; whereas, for treatment of infection, drugs arresting can be used. Systemic anti-inflammation such as ingestion of aspirin or steroids can cause several side effects such as stomach disorder and would require a larger amount than local application. Arresting, on the other hand, requires several expensive visits to the dentist. Peter can wear a dental device, for example, every night, which contains both anti-bacterial and anti-inflammation medications that are routinely prescribed for local infections and inflammation. This invention will reduce the amount of drug required for the local application, can be performed by the patient in a home setting, which will reduce overall healthcare cost and increase compliance.

Anyone familiar with the arts knows that the aforementioned drugs can be replaced by any drug or medication, which can be effectively used in the treatment of local diseases.

### EXAMPLE 3

Another example of an embodiment of the invention can be a utility for drugs that are destroyed in the gut by enzymes, low pH, or other gut conditions. Jake is suffering from cancer and needs antibody infusion for completely killing the cancerous cells. Currently, the only route of delivery for antibody or any other protein is via intravenous injections to by-pass destruction in the gut. However, Jake will be able to wear the dental or oral device as needed to deliver the antibody into his bloodstream. Since antibody molecules are large and can't be absorbed in the oral cavity, one or more drug enhancing technologies mentions elsewhere herein will be used to effectively deliver the drugs. Furthermore, often anti-cancer drugs can cause several side-effects, so oral, non-oral sensors, or other diagnostic medical devices can communicate with OMDDS such that the drug delivery can be controlled and modulated when such side-effect becomes life-threatening. This example of an embodiment of the OSWT-drug delivery system of the invention may remove the need for Jake to visit clinic or hospital for anti-cancer drug administration or delivery.

### EXAMPLE 4

In another embodiment of this invention, Charles had surgery, and his condition is stable to go home without the need for hospital care; however, his pain levels are high and intolerable. Charles can wear an OMDDS with anti-pain medication, which, depending upon need, can be activated remotely by a physician, automatically based on the pre-set program, or by Charles himself. Whenever the pain-killer is delivered to Charles; the information can be communicated to his medical records and physician who can alter post-surgery management of Charles for the best outcome and shorten his stay in the hospital. This will ensure that Charles gets relief from pain as soon as possible because the OMDDS functions as intravenous injections, which generates the fastest response to any drug or medication admissions. Therefore, Charles will require minimum pain-killer with maximum and immediate relief, which in turn will reduce the possibility of addiction to pain-killers.

In another scenario of this embodiment, the OMDDS could be linked and networked with OSWT. Severe pain can cause an increase in heart rate and elevated blood pressure, so when Charles' pain becomes severe, his heart rate and blood pressure would be elevated. These could be detected by OSWT that contain sensors for heart rate and blood pressure. When heart rate increase above the normal range of 60 to 100 beats per minute, OMDDS would deliver pain-killer medication to Charles. Such pain-killer could also be delivered by OMDDS if OSWT determines the blood pressure increase above the normal range of 120-140 MM Hg for systolic and 80-100 mm Hg for diastolic) in mm Hg.

Anyone familiar with the arts understands that many drugs and medications that need quick and fast response can be replaced in this example for a variety of diseases, disorders, and other pathological conditions.

### EXAMPLE 5

One embodiment of this invention can be used as a closed-loop insulin delivery system. A closed-loop insulin delivery system is essentially an artificial pancreas where the loop refers to the continuous cycle of feedback information such that changes in blood glucose levels can change delivered insulin in real or near time. James suffers from diabetes and needs injectable insulin when his blood sugar levels rise above normal; normal fasting glucose levels in adult humans are generally known to be between 70 and 100 mg/dL, and the non-fasting value level is about 140 mg/dL. This invention can be used in two different ways depending on the diabetic severity of James. In the first scenario, insulin can be administered in real time as the blood glucose level change; the blood glucose changes are detected by the oral or non-oral smart sensor system, and the information is continuously sent to the OMDDS-insulin, which adjusts its insulin output; and the blood glucose level changes again in response to the insulin. The loop is closed when this happens automatically. This scenario is more useful for patients with severe form of disease needing continuous blood glucose check and concomitantly insulin delivery or administration.

In the second scenario, when diabetes is milder, insulin delivery is in near-time is needed and is an example of the open or semi-closed system. When changes in the blood glucose levels are detected by the oral or non-oral smart sensor system, the information is sent to the user, James in this example, via cellular phone, computer, or another method and James can then wear the OMDDS-insulin to deliver insulin. Anyone familiar with the arts understands that this closed- or semi-closed system could use any OMDDS-insulin technologies and any oral or non-oral detecting biosensors. Similar systems for can be used for any disease related biologics and blood chemicals and therapeutic drugs with OMDDS.

These OMDDS and OSWT-OMDDS optionally are used for a variety of disease-related therapeutics including, but not limited to, local, oral and systemic infectious diseases, cancer of any phase, an oral or systemic infectious disease, drug abuse, metabolic disease, malnutrition, obesity, cardiovascular disease, atherosclerotic, infection, auto-immune and other immune diseases, stroke, AIDs, multiple sclerosis, periodontal diseases, brain-function disorders, dementia, memory loss, depression, mental disease, Alzheimer's disease, mentally-challenged disorders, nervous system disorders, and other psychology and neurological disorders, bleeding, head, neck, and other injuries, Sjogen's syndrome, oxidative stress, allergies, cancer, skeletal and muscle diseases, genetic diseases, renal diseases, osteoporosis, fatigue, stress, sleep deprivation or sleep apnea, fertility, pregnancy, ovulation, and reproductive system disorders, cystic fibrosis, respiratory or pulmonary diseases, diabetes and ketoacidosis, inflammation, age-related diseases, dehydration, halitosis, alcoholism or drug abuse, consumption or drug addiction, hypoxia, smoking-related diseases, toxins, or pollutants, poor-gait, Crohn's disease, dental caries, blood and circulatory disorders, ear, nose, and throat diseases, taste, Ageusia, Hypogeusia, or Dysgeusia, bad-breath related diseases, chewing or mastication related diseases, digestive disorders, hepatic diseases, spleen, gall-bladder and pancreatic diseases, urinary system disorders, integumentary system diseases, endocrine, lymphatic, and excretory diseases, anti-pathogen or anti-microbe, and still-to-be discovered disease-therapeutics drugs and any combination thereof.

The lack of keratinized (cornified) stratified the epithelia in the buccal and sublingual surfaces enhances solute and solvent permeability. Therefore, drug delivery via oral mucosa is more efficient as compared to delivery via the skin. Additionally, techniques that are used for transdermal drug delivery such as iontophoresis, electroporation, reverse iontophoresis, ultrasound, sonophororesis, phonophoresis, velocity based such as powder and jet injections, laser, radio frequency heating, micro-needle, tape stripping, etc. are better suited to work in the oral cavity mucosa. Therefore, these techniques along with controlled drug delivery via optimum delivery area, release time, angle, etc., as described in this invention application, offer very efficient, effective, and unique drug delivery system. These are enhancing and controlling features of the device can be controlled, turned on/off, changed, etc. manually or remotely via wireless communication parts or chips of the devices. Like esophagus, uterine, and cervix epithelia, buccal and sublingual oral mucosa epithelia, except for the dorsal surface of the tongue, is covered with a non-keratinized stratified squamous epithelium and can vary considerably in thickness in different oral regions. The dorsum of the tongue has a surface consisting of areas of both keratinized and non-keratinized epithelium; these are tightly bound to the underlying muscle of the tongue. The keratinized mucosa represents approximately 25%, the specialized mucosa (dorsum of tongue) approximately 15%, and the non-keratinized mucosa approximately 60% of the total surface area of the oral lining.

Iontophoresis and electroporation are two major means of electrically-facilitated transdermal medication administration systems. During electroporation, cells are temporarily exposed to high intensities of electric pulses that cause formation of aqueous pores in the lipid bilayers of the stratum corneum, so the drugs molecules can diffuse across skin. Using these techniques, several drug molecules can be transported across the oral mucosa with different molecular weight drugs ranging from small e.g., fentanyl, timolol, orcalcein, to high molecular weight drugs such as LHRH, calcitonin, heparin or FITC-dextran with molecular weights up to 40 kDa. Iontophoresis involves the application of physiologically acceptable electrical currents to drive charged molecules into the mucosa by electrostatic effects, and hence, ionic drugs can pass through the mucosa into the body by the created potential gradient. Unlike other trans-mucosal enhancement methodologies, it acts mainly by involving a second driving force, the electrical potential gradient as companion to the concentration gradient across the mucosa since uncharged species can also be delivered by electro-osmosis.

Thermal ablation is anther technique used for trans-mucosal drug delivery and is of two types, laser and radio frequency. The mechanism of laser thermal ablation of the mucosal is the selective removal of the stratum corneum without damaging deeper tissues, thus enhancing the delivery of lipophilic and hydrophilic drugs into mucosal layers. Lasers ablate the stratum corneum by use of optical energy and formation of micro-channels in the mucosal. Radiofrequency (RF) thermal ablation is performed by the placement of a thin, needle-like electrode directly into the mucosal and application of high frequency alternating current which produces microscopic pores in the stratum corneum and through these pores drugs can enter the body. This technique can enable trans-mucosal delivery of a wide variety of hydrophilic drugs and macromolecules.

Micro-needle array is another effective technique that can deliver drugs by minimally invasive systems. Micro-needles are multiple microscopic projections typically assembled on one side of a supporting base or patch. These micro-needles could overcome some of the disadvantages commonly associated with hypodermic needle usage and in order to address and improve patient compliance.

Velocity based devices for trans-mucosal drug delivery is of two types, powder and liquid jet injections. These employ a high-velocity jet to puncture the mucosal and deliver drugs using a power source which is either a compressed gas or a spring. There are two types of liquid jet injector single-dose jet injectors (disposable cartridge jet injectors) and multi-use-nozzle jet injectors. A jet injector is a needle free device capable of delivering electronically controlled doses of medication and they result in improved delivery consistency and reduced pain for the patient.

Additionally, these and other applications, with a variety of oral mucosa drug dispensers would yield significant information, the information without the communication and alert systems would further improve not this invention's utility to human and animal patients, caretakers, healthcare providers, and or others. This communication and alerting system, along with the oral and/or non-oral biosensors, are incorporated into the invention OSWT-oral mucosal drug delivery system. In addition to the oral and/or non-oral biosensors, the OSWT-oral drug delivery system of the invention includes other medical devices and drug dispensers that interface with the OSWT-oral mucosal drug delivery system, and are located elsewhere, within, and/or on an animal or human. The OSWT-oral mucosal drug delivery system is capable of interfacing with a variety of other medical or therapeutic devices and nano-particles that are located in or on or near the animal or human, and can securely communicate with a plurality of remote devices that monitor the health, therapeutics, and/or well-being of one or more animals or humans. The OSWT-oral mucosal drug delivery system of the invention also includes secure data system software with close and open loop system of therapeutics, visualization modes, and presentation system software. The latter feature is available to utilize a variety of platforms including, but not limited to, charts, graphs, histograms, and/or bar graphs. Caretakers, healthcare providers, etc. can visualize data including, but not limited to, correlations and comparisons between different episodes and historical data for both animal and human patients. Some of this data will be viewable and available to everyone, so will be public, yet some data will be viewable and available only to relevant individuals and healthcare providers, and hence will be private. The secure data system also inputs data into the system in order to set alerts, which can be analyzed and visualized by the OSWT-oral drug delivery software according to this aspect of the invention.

The device may be present in an oral cavity for varying amount of times. The device is optionally part of a plethora of dental or oral devices and/or it can be implanted flexibly, adhered, fixed, and/or cemented to the floor of the oral cavity. Specifically, the device is securable inside of or affixed to the cheeks, tongue, sublingual cavity, and/or other places within the oral cavity. The device can be securely placed in the oral cavity without being swallowed. The device locations within the oral cavity are optionally chosen by the patient, caretaker, and/or healthcare provider based on convenience, comfort, need, duration of placement, durability, and other factors. The drug dispenser might be placed by a healthcare provider including, but not limited to, physician, surgeon, dentist, veterinarian, healthcare provider assistant, caretaker, and/or the patient him- or herself; the placement might be done, e.g., under mild sedation, full sedation, local anesthesia, general anesthesia, or other.

The only easily accessible part of the body that is non-invasively available without a keratinized cellular barrier. Therefore, the OSWT-drug delivery system of the invention offers a unique opportunity to either quickly or sustained medication delivery for systemic and local diseases. OSWT-OMDDS drug dispensers of the invention also offer a unique opportunity to delivery medications without first-pass metabolic effects through the stomach with the acidic environment. This drug delivery could replace the invasive form of drug delivery of intravenous inj ections.

Another use is preventing narcotic prescription drug abuse. In this embodiment, real-time detection of ingested prescription medicine activates the monitoring of the time and amount of the drug consumed by the prescriber. Therefore, the OSWT-oral mucosal drug delivery system of the invention can prevent over-use or, in certain circumstances, even prevent the illegal sale of prescribed medicines. Therefore, the OSWT-OMDDS drug dispensers may optionally also function as pill trackers.

Drug administration routes are typically of two major types, non-invasive and invasive. Non-invasive can be oral, topical, and inhalation or nasal and invasive can be injections through intravenous (IV), intramuscular (IM), subcutaneous (SC), and epidural; Disadvantages of injections include potential pain or discomfort for the patient, and the requirement of trained staff using aseptic techniques for administration. The oral route, on the other hand, is the most convenient without the need for trained staff and carries the lowest cost. Oral administration is through enteral/enteric, gastric or gastrointestinal, sublingual (under the tongue) and sublabial or buccal. Some drugs can cause gastrointestinal tract irritation or can be destroyed by the gastric environment, called first-pass metabolic effects. Sublingual and sub-labial routes do not encompass first-pass metabolic effects and can mimic invasive injection route without the invasive nature of the administration route. Here we also propose administration through insertion or embedding in the oral cavity which is same as intravenous injections.

The OSWT-oral mucosal drug delivery system of the invention offers a timely, inexpensive, non-invasive or minimally invasive, and accurate medication administration route for better bio-availability. It eliminates costly, invasive, painful, and time-consuming medication administration methods. The invention's use of technologies incorporating wearable devices, devices capable of storing and analyzing large amounts of electronic data, and wireless batteries enhance the benefits provided by the OSWT-oral mucosal drug delivery system of the invention. Frequent and continuous monitoring of a large amount of data generated is readily maintained, analyzed, and handled by the inventive system and method. Moreover, the wireless charging utilized is beneficial when frequent or continuous monitoring is required.

It is to be understood that the above description is intended to be illustrative, and not restrictive. For example, the above-described embodiments (and/or aspects thereof) may be used in combination with each other. In addition, many modifications may be made to adapt a particular situation, method, system device or material to the teachings of the various embodiments of the invention without departing from their scope. While the particulars and details described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims and may include other examples that occur to those skilled in the art. Such other examples are intended to be within the scope of the claims if the examples have structural elements or steps that do not differ from the literal language of the claims, or if the examples include equivalent structural elements or steps with insubstantial differences from the literal language of the claims.

We claim:

1. A device comprising a receptacle for trans-mucosal drug delivery which is securely placed within a human or an animal's oral cavity without being swallowed.

2. The device of claim 1 wherein the receptacle is smart.

3. The device of claim 1 wherein the at least one trans-mucosal drug delivery device comprises of electrical, electroporation, ionophoresis, reverse ionophororesis, thermal ablation, laser, radio frequency heating, chemical heating, mechanical heating, vibrational, ultrasound, sonophoresis, phonophoresis, mechanical, micro-needles, tape-stripping; chemical-peels, enzyme peels, chemical enhancers, surfactants, liposomes, microemulsion, niosomes, proniosome, carrier-associate vesicles or drugs, enzyme inhibitors, nanoparticles, polymer-chain associated drugs, microparticulate, mucoadhesives, diffusion, osmosis, electro-chemical gradient assisted devices, electro-chemical gradient based, salt, natural-made polymers and compounds, man-made polymers and compound, diffusion-assisted, enzymes, polymer-chain, microparticulate, velocity-assisted devices, powder injections, jet injections and any combination thereof.

4. The device of claim 1 wherein the device is or at least one part of trans-mucosal drug delivery device is configured to have optimum contact angle, contact time, the contact area, and location for efficient drug delivery.

5. The device of claim 1 wherein the device is configured to be flexible, inflexible, fittable, moldable, adaptable, compliant, ductile, pliable, supple, tractable, tactile, transformable, workable, yielding, malleable, adjustable, changeable, conformable, convertible, bendable, and any combination thereof.

6. The device of claim 1 wherein the receptacle comprises at least one smart sensor configured to be inserted in an oral cavity of an animal or human.

7. The device of claim 1 wherein the said device is configured to be at least one interface with a network configured to utilize the information obtained from one or more extraction devices or from the one or more sensors or from one or more platforms.

8. The device of claim 1 wherein the at least one part of the said device is configured to be manually or automatically turned on or off.

9. The device of claim 1 wherein the system is configured to contain at least one of permeable, control membrane, a drug storage reservoir, software, one or more electrical input, one or more transducers configured to convert input electrical energy to different energy forms, a controller configured for providing the control signals, intensity, duration, timing, sequence, a micro-pump, and an infusion micro-pump.

10. The system of claim 1 wherein at least one of the one or more functions of the device is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible OMDDS patch, stretch stripes, adhesive stripes, OMDDS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a OMDDS gauge, OMDDS dipstick, OMDDS rod, OMDDS stick, biteplate, bendable or flexible OMDDS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent OMDDS and electronic for analytical and other functions and applications in order to provide accurate physiological information, and any combination thereof.

11. The device of the claim 1 wherein the system is configured for closed loop disease treatment.

12. The device of claim 1 wherein the one or more drugs delivered for treatment of measures cells, cancerous cells, an oral or systemic infectious disease, abuse, a metabolic disease, malnutrition, obesity, a cardiovascular disease, atherosclerotic, infection, auto-immune and other immune diseases, stroke, AIDs, multiple sclerosis, periodontal diseases, brain-function disorders, dementia, memory loss, depression, mental disease, Alzheimer's disease, mentally-challenged disorders, nervous system disorders, psychology and neurological disorders, bleeding, head and neck injuries, Sjogen's syndrome, oxidative stress, allergies, cancer, skeletal and muscle diseases, genetic diseases, diseases, osteoporosis, fatigue, stress, sleep deprivation or sleep apnea, fertility, pregnancy, ovulation, and reproductive system disorders, cystic fibrosis, respiratory or pulmonary diseases, diabetes and ketoacidosis, inflammation, age-related diseases, dehydration, halitosis, alcohol consumption, alcoholism or drug consumption or drug addiction, hypoxia, smoking-related diseases, toxins, or pollutants, poor-gait, Crohn's disease, Cushing disease, stress, dental caries, blood and circulatory disorders, ear, nose, and throat diseases, taste, Ageusia, Hypogeusia, or Dysgeusia, bad-breath related diseases chewing or mastication, digestive disorders, hepatic diseases, spleen, gall-bladder and pancreatic diseases, urinary system disorders, integumentary system diseases, endocrine, lymphatic, and excretory diseases, lymphatic, and excretory diseases.

13. The system of claim 1 wherein the system comprises at least one auxiliary smart sensor receptacle not configured to be inserted in an oral cavity of an animal or human.

14. The system of claim 1 wherein Oral Sensor Wearable Technology (OSWT) oral mucosal-OMDDS device according to the invention where the oral mucosa drug delivery system in combination with oral sensors and/or other sensors and devices are connected to “smart” medical devices in response to alerts and/or signals from the OSWT-oral mucosal drug delivery system.

15. The device of claim 1 wherein the device is configured to contain in-situ saliva component collection, concentration, isolation, storage, and communication system.

16. The device of claim 1 wherein the device is configured to contain in-situ blood component collection, concentration, isolation, storage, and communication system.

17. The device of claim 1 wherein the medication is only released when the dental device is worn by the prescribed individual.

18. The system of claim 1 comprising a network configured to provide an electronic medical records functionality.

19. The system of claim 1 wherein, the system comprises one or more units for local digital data storage independent of wireless storage, the stored data collected through any digital storage device, connector, or mechanism.

20. The device of claim 1 wherein, the means by which the receptacle is securely attached within the oral cavity of an animal or human comprises one or more of being fixedly inserted, fitted, imbedded, fitted, fixed, implanted, fastened, joined, associated, coupled, linked, banded, united, mounted, combined, glued, adhered, cemented, or firmly connected by mouthparts or hands or accessories thereof.
